(19) United States
(12) Patent Application Publication
(10) Pub. No.: US 2026/0357192 A1
(43) Pub. Date: Nov. 12, 2026

(54) INDUCIBLE AND REVERSIBLE SYSTEM AND METHODS FOR PRODUCING MORPHOTYPE-SWITCHABLE MYCOBACTERIUM ABSCESSUS STRAINS

(71) Applicants: Shelley HAYDEL, Mesa, AZ (US); Rodrigo Aguilera Olvera, Columbia, MD (US); Lexi Yakimovich, Laveen, AZ (US)

(72) Inventors: Shelley HAYDEL, Mesa, AZ (US); Rodrigo Aguilera Olvera, Columbia, MD (US); Lexi Yakimovich, Laveen, AZ (US)

(73) Assignee: ARIZONA BOARD OF REGENTS ON BEHALF OF ARIZONA STATE UNIVERSITY, Scottsdale, AZ (US)

(21) Appl. No.: 19/666,126

(22) Filed: May 1, 2026

Related U.S. Application Data
(60) Provisional application No. 63/798,534, filed on May 1, 2025.

(51) Int. Cl. C12N 1/20; C12N 9/22; C12N 15/11; C12N 15/74; C12R 1/32

(52) U.S. Cl. CPC C12N 1/20; C12N 9/226; C12N 15/111; C12N 15/74; C12N 2310/20; C12N 2830/003; C12R 2001/32

(57) ABSTRACT

Mycobacterium abscessus (Mabs) is a non-tuberculous mycobacterium that can cause severe pulmonary disease, particularly in individuals with pre-existing lung conditions, and is notoriously difficult to treat due to its intrinsic antibiotic resistance. Mabs exists in two morphotypes: smooth (Mabs^{Sm}), characterized by glycopeptidolipids (GPLs) on the cell surface, and rough (Mabs^{Rg}), which lacks GPLs and emerges during chronic infection. Disclosed herein are genetically engineered, reversible Mabs_{Sm}⇄Mabs^{Rg} morphotype switching strains developed using CRISPR interference (CRISPRi) inducible knock-down (iKD) system targeting key genes in the GPL biosynthesis and transport. Morphotype switching of these strains is induced at will by the administration of tetracycline or doxycycline, with conversion rates reaching up to 96% at 500 ng/mL ATc. The switch is reversible upon removal of ATc. These strains provide a tunable platform for modeling disease progression, studying morphotype-specific host-pathogen interactions, and screening antimicrobial agents under physiologically relevant conditions.

### RELATED APPLICATIONS

This application claims priority to and the benefit of U.S. provisional patent application 63/798,534, filed May 1, 2025, titled “Inducible and Reversible System and Methods for Producing Morphotype-Switchable Mycobacterium abscessus Strains,” the entirety of the disclosure of which is hereby incorporated by this reference.

### SEQUENCE LISTING

In accordance with 37 C.F.R. § 1.831, the present specification makes reference to a Sequence Listing submitted electronically in the form of an XML file (entitled “216US-PAT.xml”, created on May 1, 2026, 16,211 bytes in size). The entire contents of the Sequence Listing are herein incorporated by reference in their entirety, with the intention that, upon publication (including issuance), this incorporated Sequence Listing will be inserted in the published document immediately before the claims.

### TECHNICAL FIELD

This document relates to genetically modified Mycobacterium abscessus and systems of modifying M. abscessus to reduce or alter its infectious ability and virulence capacity and potential for antibiotic resistance.

### BACKGROUND

Antibiotic-resistant nontuberculous mycobacteria (NTM) infections are rising at a rate of 8% each year and account for ~$1.7 billion in annual U.S. healthcare costs. Mycobacterium abscessus (Mabs), the most common rapidly growing NTM infection, is notoriously nicknamed the “antibiotic nightmare” for its extensive intrinsic and inducible broad-range multidrug resistance to antibiotic countermeasures.

Mabs is a ubiquitous opportunistic pathogen that can cause pulmonary disease and skin and soft tissue infections. Mabs complex pulmonary disease is acquired through inhalation of Mabs-containing aerosolized water or dust. Individuals with underlying lung conditions, like cystic fibrosis (CF), are susceptible to lung colonization with Mabs and can experience recurring infections. Mabs complex skin and soft tissue infections arise from wound contact with Mabs-containing water, dust, or soil, or contaminated medical tools. Mabs displays intrinsic resistance to many common antibiotics and has limited treatment options.

The cellular stimuli underlying Mabs morphotypic switching remain largely undefined, posing a critical knowledge gap that must be addressed to advance therapeutic development and host-pathogen interaction modeling. No clinically approved antibiotic regimen reliably cures Mabs pulmonary disease. Without research and better treatments, the next two decades will bring additional clinical gaps as the U.S. population (≥65 years) ages and doubles by 2030. There is a clear and evident need to prioritize better understanding of Mabs host-pathogen interactions that correlate with infection outcomes and inform new anti-Mabs antimicrobials and therapeutic interventions.

### SUMMARY

Disclosed are genetically engineered Mycobacterium abscessus (Mabs) strains, plasmids, systems, and methods that enable inducible and reversible control of Mabs morphotype through targeted transcriptional repression of genes involved in glycopeptidolipid (GPL) biosynthesis or transport.

In one aspect, recombinant Mabs strains express a short guide RNA (sgRNA) targeting the gpl open reading frame or the gap open reading frame of Mabs. In certain embodiments, the sgRNA targets a gene selected from mmpS4, mmpL4a, and mmpL4b. In particular embodiments, sgRNAs targeting the gpl open reading frame correspond to sequences beginning between nucleotide positions 85 and 260 and have a length of 20-40 nucleotides. In specific embodiments, the sgRNA comprises a sequence set forth in SEQ ID NOs: 1-3. In other embodiments, the sgRNA targets a gene selected from mps1, mps2, and gap. In particular embodiments, sgRNAs targeting the gap open reading frame correspond to sequences beginning between nucleotide positions 110 and 150 or 350 and 400 and have a length of 15-30 nucleotides. In specific embodiments, the sgRNA comprises a sequence set forth in SEQ ID NOs: 4-6.

In another aspect, the recombinant Mabs strain comprises a CRISPR interference (CRISPRi) inducible knock-down (iKD) system. The CRISPRi system includes a catalytically inactive Cas9 (dCas9) protein and an anhydrotetracycline (ATc)-inducible TetR-regulated promoter operably linked to the sgRNA. Upon induction with ATc, expression of the sgRNA represses transcription of a targeted GPL biosynthesis or transport gene, thereby inducing a phenotypic morphotype switch from a smooth morphotype (Mabs^{Sm}) to a rough morphotype (Mabs^{condRg}). In the absence of ATc, repression is relieved and the morphotype reverts to the smooth state. The sgRNA targets the gpl open reading frame or the gap open reading frame of Mabs. In certain embodiments, the sgRNA targets a gene selected from mmpS4, mmpL4a, and mmpL4b. In particular embodiments, sgRNAs targeting the gpl open reading frame correspond to sequences beginning between nucleotide positions 85 and 260 and have a length of 20-40 nucleotides. In specific embodiments, the sgRNA comprises a sequence set forth in SEQ ID NOs: 1-3. In other embodiments, the sgRNA targets a gene selected from mps1, mps2, and gap. In particular embodiments, sgRNAs targeting the gap open reading frame correspond to sequences beginning between nucleotide positions 110 and 150 or 350 and 400 and have a length of 15-30 nucleotides. In specific embodiments, the sgRNA comprises a sequence set forth in SEQ ID NOs: 4-6.

Also disclosed are plasmids encoding a CRISPRi inducible knock-down system, the plasmids comprising a nucleotide sequence encoding an sgRNA configured to target the gpl or gap open reading frame of Mabs and a TetR-regulated promoter operably linked to expression of the sgRNA. The plasmids are configured such that administration of a tetracycline compound, including tetracycline or doxycycline, induces transcriptional repression of the targeted gene.

Further provided are systems for producing a reversible morphotype-switchable Mabs strain, comprising a CRISPRi-iKD plasmid and a tetracycline-class compound (for example, tetracycline, doxycycline, or derivative thereof). In certain embodiments, the system further comprises a Mabs strain to be transformed with the plasmid.

Methods are also disclosed for inducing a rough morphotype in Mabs by transforming the bacterium with a CRISPRi-iKD plasmid and administering a tetracycline-class compound under conditions sufficient to induce sgRNA expression and repress a GPL biosynthesis or transport gene. Methods are further disclosed for inducing reversion to a smooth morphotype by withholding the tetracycline-class compound for a period sufficient to permit re-expression of the repressed gene and restoration of the smooth morphology.

The disclosed strains, plasmids, systems, and methods provide a reversible, inducible, and genetically precise approach for controlling Mabs strain morphotype and are useful for studying morphotype-dependent biology, pathogenicity, and therapeutic responses.

The foregoing and other aspects, features, and advantages will be apparent from the DESCRIPTION and DRAWINGS, and from the CLAIMS if any are included.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The features and advantages of the present disclosure, and the manner of attaining them, will become more apparent and the present disclosure will be better understood by reference to the description of the present disclosure taken in conjunction with the accompanying drawings.

FIGS. 1A-1H depict, in accordance with certain embodiments, Mabs^{Sm} to Mabs^{condRg} morphotype switchable strains in the presence of ATc. FIGS. 1A-1C: mmpS4-mmpL4a-mmpL4b operon genes. FIGS. 1D-1F: mps1-mps2-gap operon genes. FIGS. 1G and 1H: NTsgRNA and pLJR962 empty vector control strains. Strains were induced with 10, 100, and 500 ng/ml ATc. Mabs^{Sm} CFU were smooth and glossy; in contrast, Mabs^{condRg} were flat, rough, and dry. Samples were incubated at 37 °C for 72 h. Images are representative of three biological replicates, each with 2-3 technical replicates, for all Mabs strains.

FIGS. 2A-2H depict, in accordance with certain embodiments, quantitation of Mabs^{Sm} and Mabs^{condRg} CFU in morphotype switchable strains. FIGS. 2A-2C: mmpS4-mmpL4a-mmpL4b operon genes. FIGS. 2D-2F: mps1-mps2-gap operon genes. FIGS. 2G and 2H: NTsgRNA and plJR962 empty vector control strains. Samples were incubated at 37 °C for 72 h. Quantitative results were calculated from three biological replicates, each with 2-3 technical replicates, for all Mabs strains.

FIGS. 3A-3H depict, in accordance with certain embodiments, Mabs^{condRg} reversion to Mabs^{Sm} after removal of ATc. FIGS. 3A-3C: mmpS4-mmpL4a-mmpL4b operon genes. FIGS. 3D-3F: mps1-mps2-gap operon genes. FIGS. 3G and 3H: NTsgRNA and pLJR962 empty vector control strains. Mabs^{condRg} CFU were selected and plated (indicated by arrows) onto M7H10 agar without ATc. Mabs^{condRg} CFU were flat, rough, and dry; in contrast, Mabs^{Sm} were smooth and glossy. Samples were incubated at 37 °C for 72 h.

FIGS. 4A and 4B depict, in accordance with certain embodiments, Mabs growth curves. CFU enumeration (left y-axis) and OD₆₀₀ measurements (right y-axis) of Mabs strains over 48 h in the presence (+) or absence (−) of 0.5% Tw80. Samples were grown at 37 °C for 48 h. Data represent the mean of three biological replicates, each with two technical replicates, for all Mabs strains.

FIGS. 5A-5F depict, in accordance with certain embodiments, aggregation of Mabs cultures after 15 min incubation at room temperature. Samples were grown at 37 °C in a rotary shaker for 48 h. After gentle vortexing, Mabs strains were statically incubated for 15 min at room temperature. Images are representative of three biological replicates for all Mabs strains.

FIG. 6 depicts, in accordance with certain embodiments, aggregation aggregative index of Mabs strains at 48 hours. Quantitative results were calculated from three biological replicates, each with two technical replicates, for all Mabs strains. Statistical analysis was completed using two-way ANOVA with multiple comparisons. ****, p<0.001.

FIGS. 7A-7H depict, in accordance with certain embodiments, aggregation of Mabs^{condRg} cultures after gentle vortexing. FIGS. 7A-7C: mmpS4-mmpL4a-mmpL4b operon genes. FIGS. 7D-7F: mps1-mps2-gap operon genes. FIGS. 7G and 7H: NTsgRNA and pLJR962 empty vector control strains. Samples were grown at 37 °C in a rotary shaker for 48 h, with ATc supplementation at 24 h. After removal from incubation, samples were gently vortexed. Images are representative of three biological replicates for all Mabs strains.

FIGS. 8A-8H depict, in accordance with certain embodiments, aggregation of Mabs^{condRg} cultures after 15 min incubation at room temperature. FIGS. 8A-8C: mmpS4-mmpL4a-mmpL4b operon genes. FIGS. 8D-8F: mps1-mps2-gap operon genes. FIGS. 8G and 8H: NTsgRNA and pLJR962 empty vector control strains. Samples were grown at 37 °C in a rotary shaker for 48 h, with ATc supplementation at 24 h. After gentle vortexing, Mabs strains were statically incubated for 15 min at room temperature. Images are representative of three biological replicates for all Mabs strains.

FIGS. 9A and 9B depict, in accordance with certain embodiments, aggregative index of Mabs^{condRg} strains at 48 h. Quantitative results were calculated from three biological replicates for all Mabs^{condRg} strains. FIG. 9A: mmpS4-mmpL4a-mmpL4b operon genes with NTsgRNA and pLJR962 empty vector control strains. FIG. 9B: mps1-mps2-gap operon genes with NTsgRNA and pLJR962 empty vector control strains. Supplementation with 0.05% or 0.5% Tween 80 (Tw80) is indicated with red squares or black circles, respectively. Absence of Tw80 is denoted with blue triangles.

FIG. 10 depicts, in accordance with certain embodiments, the vector map of pLJR962, the sequence of which is set forth in SEQ ID NO: 7.

### DETAILED DESCRIPTION

Detailed aspects and applications of the disclosure are described below in the following drawings and detailed description of the technology. Unless specifically noted, it is intended that the words and phrases in the specification and the claims be given their plain, ordinary, and accustomed meaning to those of ordinary skill in the applicable arts.

In the following description, and for the purposes of explanation, numerous specific details are set forth in order to provide a thorough understanding of the various aspects of the disclosure. It will be understood, however, by those skilled in the relevant arts, that embodiments of the technology disclosed herein may be practiced without these specific details. It should be noted that there are many different and alternative configurations, devices and technologies to which the disclosed technologies may be applied. The full scope of the technology disclosed herein is not limited to the examples that are described below.

The singular forms “a,” “an,” and “the” include plural referents unless the context clearly dictates otherwise. Thus, for example, reference to “a step” includes reference to one or more of such steps.

The word “exemplary,” “example,” or various forms thereof are used herein to mean serving as an example, instance, or illustration. Any aspect or design described herein as “exemplary” or as an “example” is not necessarily to be construed as preferred or advantageous over other aspects or designs. Furthermore, examples are provided solely for purposes of clarity and understanding and are not meant to limit or restrict the disclosed subject matter or relevant portions of this disclosure in any manner. It is to be appreciated that a myriad of additional or alternate examples of varying scope could have been presented but have been omitted for purposes of brevity.

As used herein, the term “tetracycline-class compound” refers to any natural, semi-synthetic, or fully synthetic compound that contains the tetracycline core scaffold, namely the octahydro-naphthacene-2-carboxamide skeleton. Such compounds include but are not limited to tetracycline, oxytetracycline, chlortetracycline, doxycycline, minocycline, tigecycline, and their derivatives.

When a range of values is expressed, another embodiment includes from the one particular value and/or to the other particular value. Similarly, when values are expressed as approximations, by use of the antecedent “about,” it will be understood that the particular value forms another embodiment. All ranges are inclusive and combinable.

Throughout the description and claims of this specification, the words “comprise” and “contain” and variations of the words, for example “comprising” and “comprises”, mean “including but not limited to”, and are not intended to (and do not) exclude other components.

As required, detailed embodiments of the present disclosure are included herein. It is to be understood that the disclosed embodiments are merely exemplary of the invention that may be embodied in various forms. Therefore, specific structural and functional details disclosed herein are not to be interpreted as limits, but merely as a basis for teaching one skilled in the art to employ the present invention. The specific examples below will enable the disclosure to be better understood. However, they are given merely by way of guidance and do not imply any limitation.

The present disclosure may be understood more readily by reference to the following detailed description taken in connection with the accompanying figures and examples, which form a part of this disclosure. It is to be understood that this disclosure is not limited to the specific materials, devices, methods, applications, conditions, or parameters described and/or shown herein, and that the terminology used herein is for the purpose of describing particular embodiments by way of example only and is not intended to be limiting of the claimed inventions. The term “plurality”, as used herein, means more than one. When a range of values is expressed, another embodiment includes from the one particular value and/or to the other particular value. Similarly, when values are expressed as approximations, by use of the antecedent “about,” it will be understood that the particular value forms another embodiment. All ranges are inclusive and combinable.

As part of its natural infection cycle, Mycobacterium abscessus (Mabs) undergoes a morphotypical conversion from smooth (Mabs^{Sm}) to rough (Mabs^{Rg}). This morphotypic conversion is intimately associated with increased macrophage invasion and disease progression, ultimately leading to debilitating, refractory Mabs pulmonary disease. The two morphotypes are differentiated based on the presence (Mabs^{Sm}) or absence (Mabs^{Rg}) of glycopeptidolipids (GPLs) coating the cell envelope. Mabs^{Sm} is passively motile (via sliding), biofilm-forming, and believed to be the infecting form of Mabs, while Mabs^{Rg} is non-motile, non-biofilm-forming, and can cord. Mabs^{Rg} is also associated with increased resistance to antibiotics and phagocytosis. The Mabs^{Sm}-to-Mabs^{Rg} morphotype switch can occur after years of infection due to unknown triggers. Specific stimuli triggering Mabs morphotypical conversion are unknown, thus preventing directed investigations into morphotype-specific immunological responses and the discovery of morphotype-specific therapeutic targets.

GPLs are an important virulence factors during intracellular infection. When Mabs^{Sm} is phagocytosed by macrophages, GPLs interact with the cell membrane of the macrophage to inhibit apoptosis by suppressing the release of cytochrome c and radical oxygen species (ROS) production. In addition, GPLs interact with cyclophilin D, causing inhibition of the mitochondrial permeability transition pore and allowing Mabs to survive within the macrophage.

Mabs operons mmpS4-mmpL4a-mmpL4b and mbtH-mps1-mps2-gap are important for GPL expression. MmpS4-mmpL4a-mmpL4b encodes transmembrane proteins MmpS4, MmpL4a, and Mmpl4b, respectively, which are important for GPL transport. The mbtH-mps1-mps2-gap operon encodes the proteins MbtH, Mps1, Mps2, and Gap. Mabs Mps1 and Mps2 are peptide synthetases that are required for GPL biosynthesis, and Gap is a transmembrane protein required for GPL transport and biosynthesis. If one gene in either of the operons is disrupted, a loss of GPLs occurs, and the bacterial morphotype transitions from Mabs^{Sm} to M. Mabs^{Rg}.

A system using molecular genetic tools, including CRISPR interference and inducible knockdown control of CRISPRi via a tetracycline/doxycycline-inducible TetR-regulated promoter-operator system is disclosed for creating a reversible Mabs smooth to conditional rough morphotype molecular toolbox. CRISPR interference (CRISPRi) inducible knock-down (iKD) system for M. tuberculosis and M. smegmatis has been adapted for transcriptional repression of Mabs genes. Using the mycobacterial CRISPRi system, a novel, reversible Mabs^{Sm}↔Mabs^{Rg} morphotype switchable strains was created by targeting the genes of the mmpS4-mmpL4a-mmpL4b and mps1-mps2-gap operons.

The CRISPRi-iKD system may be based on either one of the two classes of CRISPR-Cas systems. Class 1 CRISPR-Cas systems have effector modules composed of multiple Cas proteins, some of which form crRNA-binding complexes, while Class 2 CRISPR-Cas systems include a single, multi-domain crRNA-binding protein. In some embodiments, the CRISPR-Cas system that can be used to modify a polynucleotide of the present invention described herein can be a Class 1 CRISPR-Cas system. In some embodiments, the CRISPR-Cas system that can be used to modify a polynucleotide of the present invention described herein can be a Class 2 CRISPR-Cas system.

### Class 1 CRISPR-Cas Systems

In some embodiments, the CRISPR-Cas system that can be used to modify a polynucleotide of the present invention described herein can be a Class 1 CRISPR-Cas system. Class 1 CRISPR-Cas systems are divided into types I, II, and IV. Type I CRISPR-Cas systems are divided into 9 subtypes (I-A, I-B, I-C, I-D, I-E, I-F1, I-F2, I-F3, and IG). Class 1, Type I CRISPR-Cas systems can contain a Cas3 protein that can have helicase activity. Type III CRISPR-Cas systems are divided into 6 subtypes (III-A, III-B, III-C, III-D, III-E, and III-F). Type III CRISPR-Cas systems can contain a Cas10 that can include an RNA recognition motif called Palm and a cyclase domain that can cleave polynucleotides. Type IV CRISPR-Cas systems are divided into 3 subtypes. (IV-A, IV-B, and IV-C). Class 1 systems also include CRISPR-Cas variants, including Type I-A, I-B, I-E, I-F and I-U variants, which can include variants carried by transposons and plasmids, including versions of subtype I-F encoded by a large family of Tn7-like transposon and smaller groups of Tn7-like transposons that encode similarly degraded subtype I-B systems.

The Class 1 systems typically comprise a multi-protein effector complex, which can, in some embodiments, include ancillary proteins, such as one or more proteins in a complex referred to as a CRISPR-associated complex for antiviral defense (Cascade), one or more adaptation proteins (e.g., Cas1, Cas2, RNA nuclease), and/or one or more accessory proteins (e.g., Cas 4, DNA nuclease), CRISPR associated Rossman fold (CARF) domain containing proteins, and/or RNA transcriptase.

The backbone of the Class 1 CRISPR-Cas system effector complexes can be formed by RNA recognition motif domain-containing protein(s) of the repeat-associated mysterious proteins (RAMPs) family subunits (e.g., Cas 5, Cas6, and/or Cas7). RAMP proteins are characterized by having one or more RNA recognition motif domains. In some embodiments, multiple copies of RAMPs can be present. In some embodiments, the Class I CRISPR-Cas system can include 1, 2, 3, 4, 5, 6, 7, 8, 9, 10, 11, 12 or more Cas5, Cas6, and/or Cas 7 proteins. In some embodiments, the Cas6 protein is an RNAse, which can be responsible for pre-crRNA processing. When present in a Class 1 CRISPR-Cas system, Cas6 can be optionally physically associated with the effector complex.

Class 1 CRISPR-Cas system effector complexes can, in some embodiments, also include a large subunit. The large subunit can be composed of or include a Cas8 and/or Cas10 protein.

Class 1 CRISPR-Cas system effector complexes can, in some embodiments, include a small subunit (for example, Cas11).

In some embodiments, the Class 1 CRISPR-Cas system can be a Type I CRISPR-Cas system. In some embodiments, the Type I CRISPR-Cas system can be a subtype I-A CRISPR-Cas system. In some embodiments, the Type I CRISPR-Cas system can be a subtype I-B CRISPR-Cas system. In some embodiments, the Type I CRISPR-Cas system can be a subtype I-C CRISPR-Cas system. In some embodiments, the Type I CRISPR-Cas system can be a subtype I-D CRISPR-Cas system. In some embodiments, the Type I CRISPR-Cas system can be a subtype I-E CRISPR-Cas system. In some embodiments, the Type I CRISPR-Cas system can be a subtype I-F1 CRISPR-Cas system. In some embodiments, the Type I CRISPR-Cas system can be a subtype I-F2 CRISPR-Cas system. In some embodiments, the Type I CRISPR-Cas system can be a subtype I-F3 CRISPR-Cas system. In some embodiments, the Type I CRISPR-Cas system can be a subtype I-G CRISPR-Cas system. In some embodiments, the Type I CRISPR-Cas system can be a CRISPR Cas variant, such as a Type I-A, I-B, I-E, I-F and I-U variants, which can include variants carried by transposons and plasmids, including versions of subtype I-F encoded by a large family of Tn7-like transposon and smaller groups of Tn7-like transposons that encode similarly degraded subtype I-B systems.

In some embodiments, the Class 1 CRISPR-Cas system can be a Type III CRISPR-Cas system. In some embodiments, the Type III CRISPR-Cas system can be a subtype III-A CRISPR-Cas system. In some embodiments, the Type III CRISPR-Cas system can be a subtype III-B CRISPR-Cas system. In some embodiments, the Type III CRISPR-Cas system can be a subtype III-C CRISPR-Cas system. In some embodiments, the Type III CRISPR-Cas system can be a subtype III-D CRISPR-Cas system. In some embodiments, the Type III CRISPR-Cas system can be a subtype III-E CRISPR-Cas system. In some embodiments, the Type III CRISPR-Cas system can be a subtype III-F CRISPR-Cas system.

In some embodiments, the Class 1 CRISPR-Cas system can be a Type IV CRISPR-Cas-system. In some embodiments, the Type IV CRISPR-Cas system can be a subtype IV-A CRISPR-Cas system. In some embodiments, the Type IV CRISPR-Cas system can be a subtype IV-B CRISPR-Cas system. In some embodiments, the Type IV CRISPR-Cas system can be a subtype IV-C CRISPR-Cas system.

The effector complex of a Class 1 CRISPR-Cas system can, in some embodiments, include a Cas3 protein that is optionally fused to a Cas2 protein, a Cas4, a Cas5, a Cas6, a Cas7, a Cas8, a Cas10, a Cas11, or a combination thereof. In some embodiments, the effector complex of a Class 1 CRISPR-Cas system can have multiple copies, such as 1, 2, 3, 4, 5, 6, 7, 8, 9, 10, 11, 12, 13, or 14, of any one or more Cas proteins.

### Class 2 CRISPR-Cas Systems

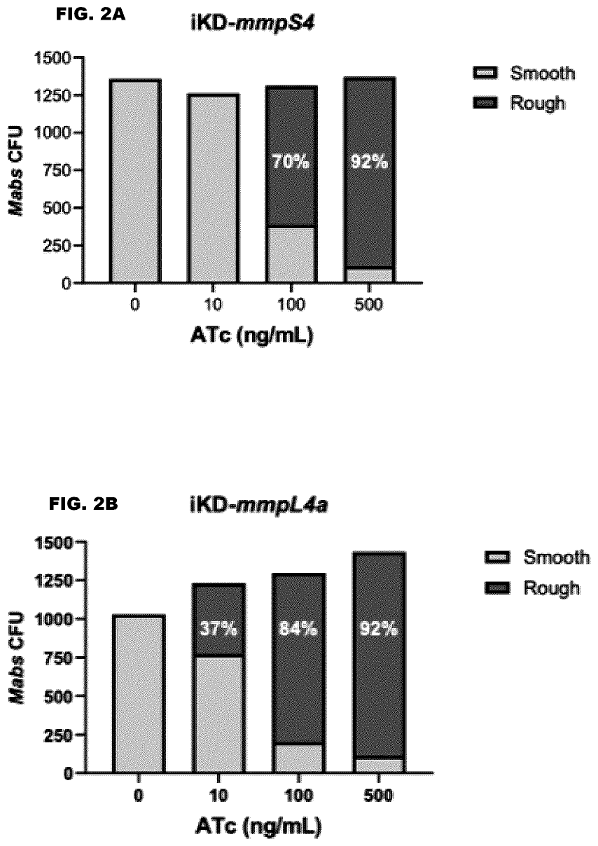

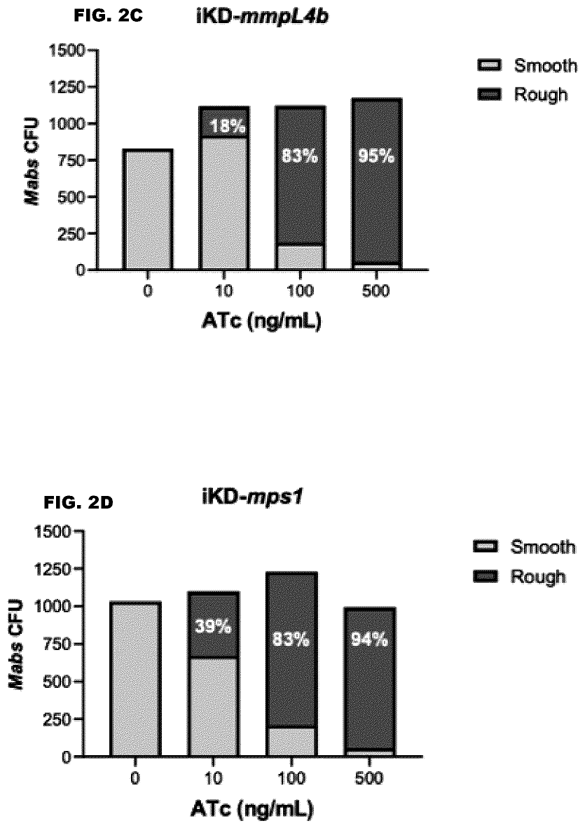

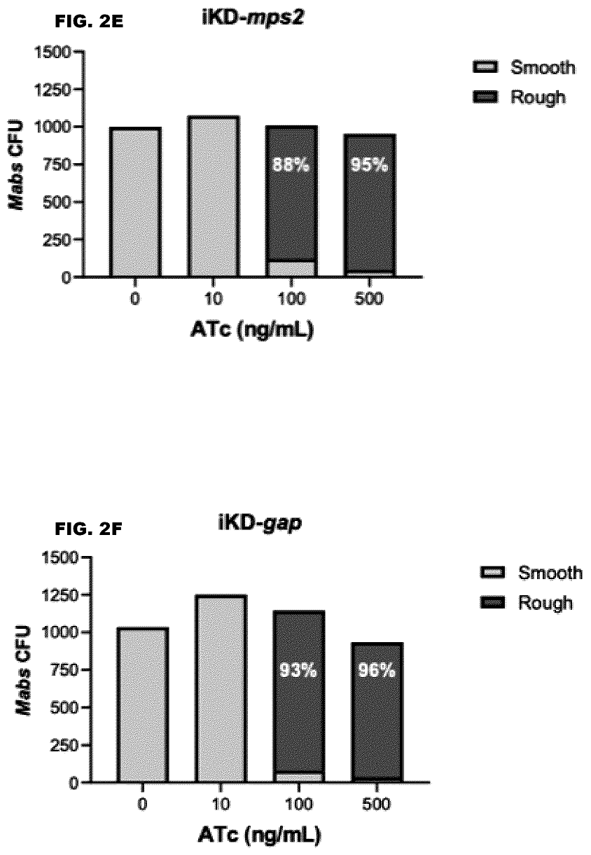

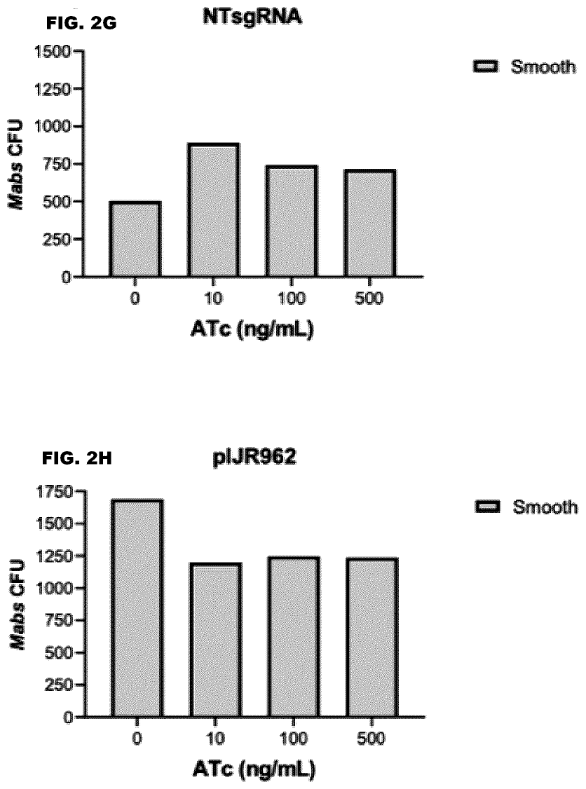

The compositions, systems, and methods described in greater detail elsewhere herein can be designed and adapted for use with Class 2 CRISPR-Cas systems. Thus, in some embodiments, the CRISPR-Cas system is a Class 2 CRISPR-Cas system. Class 2 systems are distinguished from Class 1 systems in that they have a single, large, multi-domain effector protein. In certain example embodiments, the Class 2 system can be a Type II, Type V, or Type VI system, which are described in Makarova et al. “Evolutionary classification of CRISPR-Cas systems: a burst of class 2 and derived variants” Nature Reviews Microbiology, 18:67-81 (February 2020), incorporated herein by reference. Each type of Class 2 system is further divided into subtypes. See Markova et al. 2020, particularly at Figure. 2. Class 2, Type II systems can be divided into 4 subtypes: II-A, II-B, II-C1, and II-C2. Class 2, Type V systems can be divided into 17 subtypes: V-A, V-B1, V-B2, V-C, V-D, V-E, V-F1, V-F1 (V-U3), V-F2, V-F3, V-G, V-H, V-I, V-K (V-U5), V-U1, V-U2, and V-U4. Class 2, Type IV systems can be divided into 5 subtypes: VI-A, VI-B1, VI-B2, VI-C, and VI-D.

The distinguishing feature of these types is that their effector complexes consist of a single, large, multi-domain protein. Type V systems differ from Type II effectors (e.g., Cas9), which contain two nuclear domains that are each responsible for the cleavage of one strand of the target DNA, with the HNH nuclease inserted inside the Ruv-C like nuclease domain sequence. The Type V systems (e.g., Cas12) only contain a RuvC-like nuclease domain that cleaves both strands. Type VI (Cas13) are unrelated to the effectors of Type II and V systems and contain two HEPN domains and target RNA. Cas13 proteins also display collateral activity that is triggered by target recognition. Some Type V systems have also been found to possess this collateral activity with two single-stranded DNA in in vitro contexts.

In some embodiments, the Class 2 system is a Type II system. In some embodiments, the Type II CRISPR-Cas system is a II-A CRISPR-Cas system. In some embodiments, the Type II CRISPR-Cas system is a II-B CRISPR-Cas system. In some embodiments, the Type II CRISPR-Cas system is a II-C1 CRISPR-Cas system. In some embodiments, the Type II CRISPR-Cas system is a II-C2 CRISPR-Cas system. In some embodiments, the Type II system is a Cas9 system. In some embodiments, the Type II system includes a Cas9.

In some embodiments, the Class 2 system is a Type V system. In some embodiments, the Type V CRISPR-Cas system is a V-A CRISPR-Cas system. In some embodiments, the Type V CRISPR-Cas system is a V-B1 CRISPR-Cas system. In some embodiments, the Type V CRISPR-Cas system is a V-B2 CRISPR-Cas system. In some embodiments, the Type V CRISPR-Cas system is a V-C CRISPR-Cas system. In some embodiments, the Type V CRISPR-Cas system is a V-D CRISPR-Cas system. In some embodiments, the Type V CRISPR-Cas system is a V-E CRISPR-Cas system. In some embodiments, the Type V CRISPR-Cas system is a V-F1 CRISPR-Cas system. In some embodiments, the Type V CRISPR-Cas system is a V-F1 (V-U3) CRISPR-Cas system. In some embodiments, the Type V CRISPR-Cas system is a V-F2 CRISPR-Cas system. In some embodiments, the Type V CRISPR-Cas system is a V-F3 CRISPR-Cas system. In some embodiments, the Type V CRISPR-Cas system is a V-G CRISPR-Cas system. In some embodiments, the Type V CRISPR-Cas system is a V-H CRISPR-Cas system. In some embodiments, the Type V CRISPR-Cas system is a V-I CRISPR-Cas system. In some embodiments, the Type V CRISPR-Cas system is a V-K (V-U5) CRISPR-Cas system. In some embodiments, the Type V CRISPR-Cas system is a V-U1 CRISPR-Cas system. In some embodiments, the Type V CRISPR-Cas system is a V-U2 CRISPR-Cas system. In some embodiments, the Type V CRISPR-Cas system is a V-U4 CRISPR-Cas system. In some embodiments, the Type V CRISPR-Cas system includes a Cas12a (Cpf1), Cas12b (C2c1), Cas12c (C2c3), Cas12d (CasY), Cas12e (CasX), Cas14, and/or CasΦ.

In some embodiments the Class 2 system is a Type VI system. In some embodiments, the Type VI CRISPR-Cas system is a VI-A CRISPR-Cas system. In some embodiments, the Type VI CRISPR-Cas system is a VI-B1 CRISPR-Cas system. In some embodiments, the Type VI CRISPR-Cas system is a VI-B2 CRISPR-Cas system. In some embodiments, the Type VI CRISPR-Cas system is a VI-C CRISPR-Cas system. In some embodiments, the Type VI CRISPR-Cas system is a VI-D CRISPR-Cas system. In some embodiments, the Type VI CRISPR-Cas system includes a Cas13a (C2c2), Cas13b (Group 29/30), Cas13c, and/or Cas13d.

In certain implementations, the CRISPRi-iKD system comprises a catalytically inactive Cas9 (dCas9) protein and a tetracycline/doxycycline-inducible TetR-regulated promoter operably linked to a sgRNA. The sgRNA targets a gene selected from the group consisting of mmpS4, mmpL4a, mmpL4b, mps1, mps2, and gap, such that, upon induction with tetracycline or doxycycline, expression of the sgRNA represses the target gene, inducing a phenotypic morphotype switch from a smooth morphotype (Mabs^{Sm}) to a rough morphotype (Mabs^{condRg}), and in the absence of tetracycline or doxycycline, the morphotype reverts to Mabs^{Sm}.

Mycobacterial CRISPR interference regulates the expression of Mabs surface GPLs to result in repression and de-repression of six independent GPL genetic targets. Due to this, morphological switching can occur and be reversible. By controlling surface GPLs, Mabs infectious processes can be modulated without permanently manipulating the genome or molecular pathways. The Mabs morphoswitching molecular toolbox disclosed herein controls Mabs^{condRg} morphotype switching at-will upon induction of tetracycline (for example, anhydrotetracycline, ATc) and reversion to Mabs^{Sm} in the absence of tetracycline. In other words, the Mabs^{Sm}↔Mabs^{Rg} morphotype switchable strains generated can induce the morphotype switch at-will with tetracycline and revert in the absence of tetracycline. These strains are novel molecular genetic tools that can be used to better understand M. abscessus physiology, virulence mechanisms, and host-cell interactions during intracellular infection. These strains can also be utilized for more clinically predictive animal studies by accurately simulating Mabs disease pathology with the morphotype switch. These strains advance the development of more physiologically relevant in vitro and in vivo models and supports the discovery of more effective therapeutic strategies.

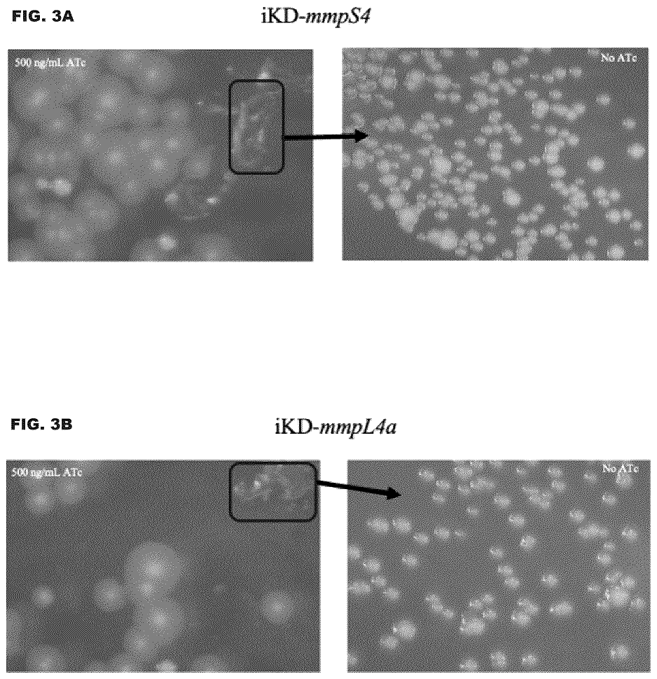

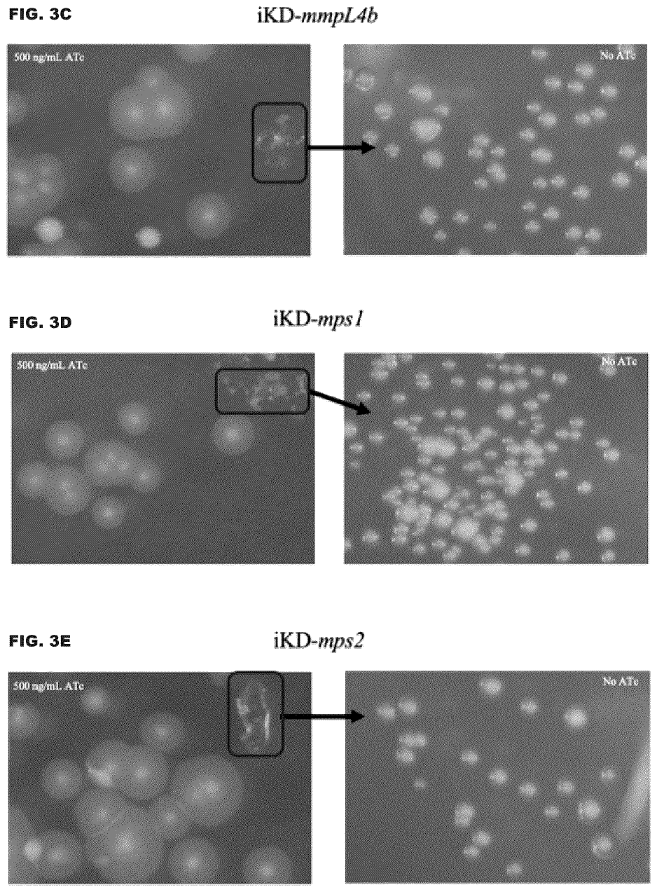

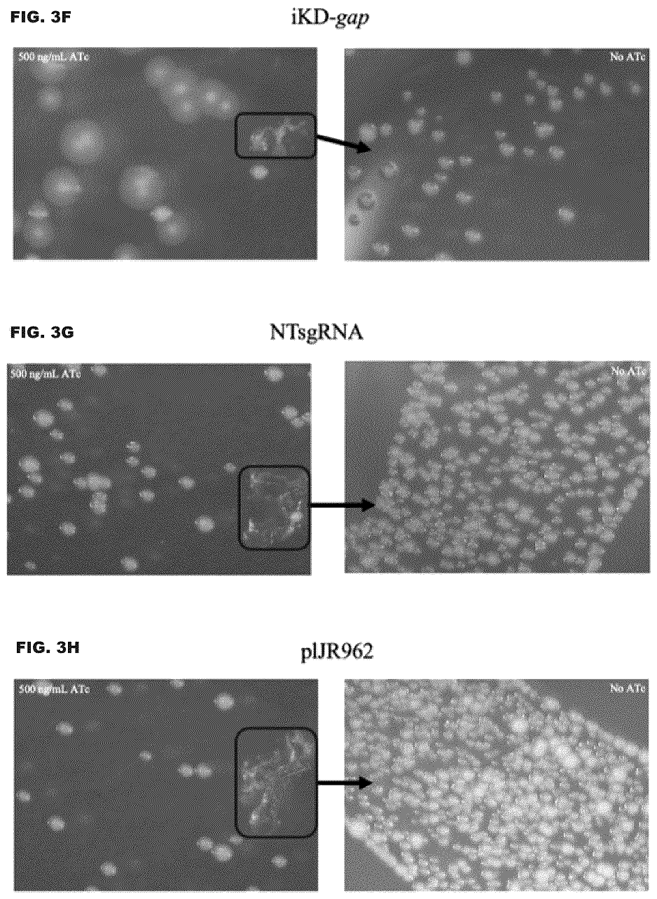

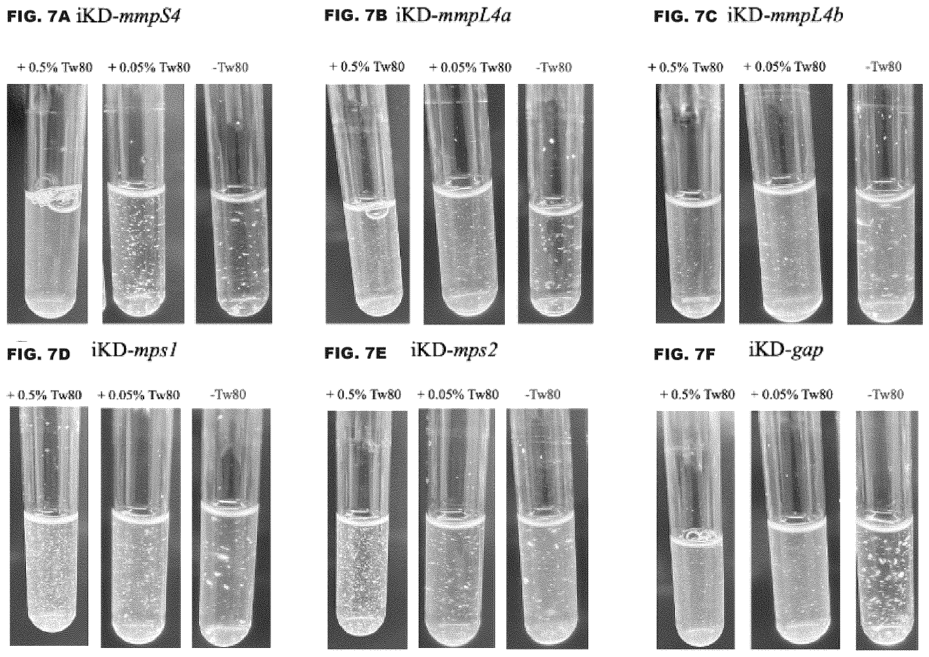

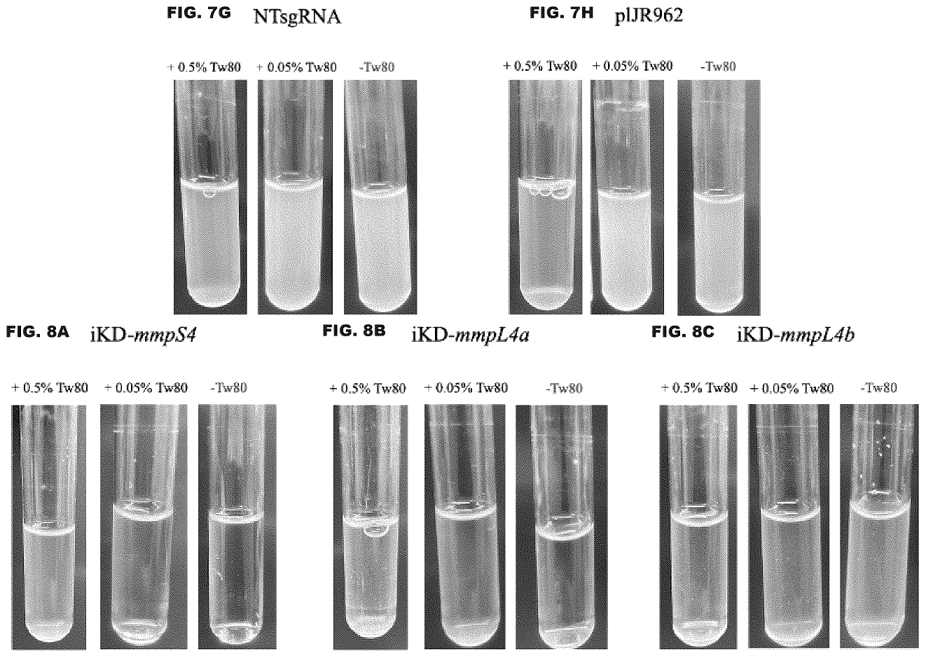

As shown in the Examples, 500 ng/mL ATc induced the highest and most consistent Mabs^{condRg} conversion rate (between 92-96%) for all morphoswitching strains (FIGS. 2A-2H). The desired Mabs^{Sm}. Mabs^{condRg} ratio can be attained by adjusting the concentration of ATc. The strains are morphotypic reversible in the absence of ATc (FIGS. 3A-3H). In addition, the aggregative index was determined to sufficiently differentiate and quantitate the Mabs^{Sm} and Mabs^{condRg} morphotype switching in liquid culture (FIGS. 7A-7H).

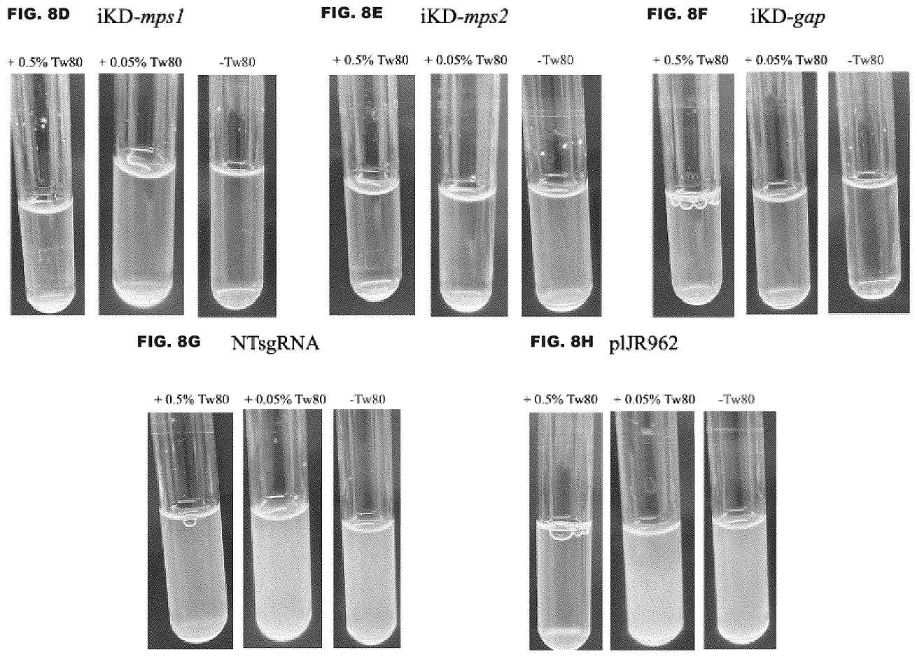

Aggregation profiling was used to confirm the maintained Mabs^{condRg} morphotype upon continuous induction with ATc (FIGS. 7A-8H). To maintain the rough morphology, it is important the morphoswitching strains are in the presence of a tetracycline compound. ATc's half-life is ~20 h, so to maintain the morphotype switch, ATc needs to be dosed every 24 h. ATc is temperature- and light-sensitive, which routinely caused challenges during bacterial plating, but the mycobacterial CRISPRi system is also inducible by doxycycline. Doxycycline is less light-sensitive than ATc and therefore could be an alternative; though it has a half-life of ~18-22 h, so being dosed every 24 h or similar dosing strategies as ATc would still be necessary. Accordingly, the use of ATc versus doxycycline depends on the dosing requirement. For the Examples, ATc was used as the inducer due to its effectiveness at lower doses. An alternative strategy for long-term in vivo testing to prevent the need for daily ATc dosage is using CRISPRi to perform a gene knock-out on mps1 with CRISPR/Cas9. However, this resulting Mabs strain lacks the ability to reverse morphology switching.

The Mabs morphoswitching molecular toolbox represents a significant advancement for the study of Mabs-associated diseases, particularly in the context of Mabs complex pulmonary disease. It advances the development of more physiologically relevant in vitro and in vivo models, enhances our ability to dissect cellular mechanisms underlying morphotype transitions, and supports the discovery of more effective therapeutic strategies. This versatile system enables precise manipulation, control, and tracking of morphotype transitions, which are central to Mabs pathogenicity, antibiotic tolerance, and immune evasion. As such, it provides an essential platform for high-throughput screening of novel antimicrobial agents and rational design of combination therapies targeting morphotype-specific vulnerabilities. Furthermore, the toolbox facilitates the development of more physiologically relevant and more clinically predictive preclinical models, including murine models that better mimic the dynamics of Mabs infection in humans. These improved models will be critical for evaluating drug efficacy and host-pathogen interactions in a more translationally meaningful context.

The Mabs morphoswitching toolbox also establishes a foundation for improved preclinical drug development. By leveraging this novel platform for drug screening and therapeutic discovery, we can investigate and exploit morphotype-specific susceptibilities and host-pathogen interactions both in vitro and in vivo. By facilitating tunable, controlled manipulation of Mabs morphotypes, the molecular toolbox supports the identification of morphotype-dependent pathways that can be selectively targeted by new or repurposed therapeutics.

Also described are recombinant Mabs strains that exhibit switchable morphology. In some embodiments, the recombinant Mabs expresses a sgRNA targeting the gpl open reading frame of Mabs. In some aspects, the sgRNA targets a gene selected from the group consisting of: mmpS4, mmpL4a, and mmpL4b. In certain implementation, the sgRNA corresponds with a sequence on the gpl open reading frame starting at position 85-260 and have a length of 20-40 nucleotides. For example, the sgRNA has a sequence set forth in any one of SEQ ID NOs: 1-3. In other embodiments, the recombinant Mabs expresses a sgRNA targeting the gap open reading frame of Mabs. In some aspects, the sgRNA targets a gene selected from the group consisting of: mps1, mps2, and gap. In certain implementations, the sgRNA corresponds with a sequence on the gap open reading frame starting at position 110-150 or starting at position 350-400 and have a length of 15-30 nucleotides. For example, the sgRNA has a sequence set forth in any one of SEQ ID NOs: 4-6. Expression of the sgRNA in the recombinant Mabs is controlled by an inducible promoter, for example, a TetR-regulated promoter. In certain implementations, the sgRNA is expressed by a plasmid encoding the CRISPRi-iKD system where the CRISPRi-iKD system comprises an inducible promoter for expressing the sgRNA. In some aspects, the inducible promoter for expressing the sgRNA is a TetR-regulated promoter. Thus, recombinant Mabs comprising a CRISPRi system is also described herein.

Plasmids encoding the Mabs morphoswitching molecular toolbox are also described. The plasmid comprises a sequence encoding a sgRNA configured to target the gpl open reading frame or the gap open reading frame of Mabs and an inducible promoter operably linked to expression of the sgRNA. In some aspects, the inducible promoter for expressing the sgRNA is a TetR-regulated promoter. The plasmid is configured to induce transcriptional repression of the targeted gene upon administration of a tetracycline-class compound. The tetracycline-class compound may be selected from tetracycline, doxycycline, or a derivative thereof. In some aspects, the sgRNA targets a gene selected from the group consisting of: mmpS4, mmpL4a, and mmpL4b. For example, the sgRNA has a sequence set forth in any one of SEQ ID NOs: 1-3. In other embodiments, the recombinant Mabs expresses a sgRNA targeting the gap open reading frame of Mabs. In some aspects, the sgRNA targets a gene selected from the group consisting of: mps1, mps2, and gap. For example, the sgRNA has a sequence set forth in any one of SEQ ID NOs: 4-6. In another aspect, a system for producing a reversible morphotype switchable strain of Mabs is described. The system comprises the plasmid encoding the Mabs morphoswitching molecular toolbox and a tetracycline-class compound. In some implementations, the system further comprises a Mabs strain.

Kits for producing a reversible morphotype switchable strain of Mabs and for inducing a specific morphology in Mabs are additionally disclosed. In some embodiments, the kit for producing a reversible morphotype switchable strain of Mabs comprises the plasmid described herein and instructions for inducing the rough morphology and reversing to smooth morphology. In some aspects, the instructions provide that administering an inducer of the inducible promoter to Mabs transformed with the plasmid under conditions sufficient to activate expression of an sgRNA and repress expression of a GPL biosynthesis or transport gene induces a rough morphotype in the strain. In some aspects, the instructions also provide that withholding administration and exposure of the transformed Mabs to the inducer permits re-expression of the gene targeted by the sgRNA and reversion of the Mabs to a smooth morphology. In some implementations where the plasmid comprise is a TetR-regulated promoter for expressing the sgRNA, the inducer is a tetracycline-class compound such as tetracycline, doxycycline, or a derivative thereof. In some embodiments, the kit can further comprise a Mabs strain and/or a tetracycline-class compound.

The kit for inducing a specific morphology of Mabs comprises the recombinant Mabs strain that exhibit switchable morphology described herein and instructions for inducing the rough morphology and reversing to smooth morphology. In some aspects, the instructions provide that administering an inducer of the inducible promoter recombinant Mabs strain under conditions sufficient to activate expression of an sgRNA and repress expression of a GPL biosynthesis or transport gene induces a rough morphotype in the strain. In some aspects, the instructions also provide that withholding administration and exposure of the transformed Mabs to the inducer permits re-expression of the gene targeted by the sgRNA and reversion of the Mabs to a smooth morphology. In some implementations, the kit further comprises the inducer. In some certain embodiments, the inducer is a tetracycline-class compound such as tetracycline, doxycycline, or a derivative thereof.

Further disclosed are a method of inducing Mabs morphology change to a rough morphology and a method of inducing Mabs morphology change to a smooth morphology. To induce Mabs morphology change to a rough morphology, the method comprises transforming the Mabs with a plasmid encoding the Mabs morphoswitching molecular toolbox described herein; and administering an inducer (for example, a tetracycline-class compound) to the transformed Mabs under conditions sufficient to activate expression of an sgRNA and repress expression of a GPL biosynthesis or transport gene, thereby inducing a rough morphotype in the strain. In some implementations where the sgRNA expression is regulated by a TetR-regulated promoter, the inducer is readministered to the transformed Mabs at least one every 24 hours, for example, once every 18-22 hours. To induce Mabs morphology change to a smooth morphology, the method comprises providing a transformed Mabs induced to have a rough morphology and then withholding administration and exposure of the transformed Mabs to the inducer to permit re-expression of the gene targeted by the sgRNA and reversion of the Mabs to a smooth morphology. In certain implementations, the method of inducing Mabs morphology change to a smooth morphology comprises withholding administration and exposure of the transformed Mabs to a tetracycline-class compound for at least 18 hours.

### EXAMPLES

The present disclosure is further illustrated by the following examples that should not be construed as limiting.

### Example 1: Target GPL Operons Using CRISPRi-iKD to Create Novel, Reversible Mabs^{Sm}↔Mabs^{Rg} Morphotype Switchable Strains

The Mabs morphoswitching molecular toolbox was developed using CRISPRi and ATc-inducible TetR-regulated promoter iKD control (Ehrt, “Controlling gene expression in mycobacteria with anhydrotetracycline and Tet repressor.” Nucleic Acids Research 2005, 33 (2), e21-e21; Rock, et al. “Programmable transcriptional repression in mycobacteria using an orthogonal CRISPR interference platform.” Nat Microbiol 2017, 2 (4), 16274) to conditionally repress and de-repress GPL genetic targets within the mmpS4-mmpL4 or mps-gap operons. Six recombinant Mabs strains were generated, specifically targeting the mmpS4, mmpL4a, mmpL4b, mps1, mps2, or gap genes. The mmpS4-mmpL4a-mmpL4b operon is involved in GPL transport, and mps1-mps2-gap is involved in GPL biosynthesis (mps1, mps2) and transport (gap). The PAM sequence, sgRNA targeting sequence, and predicted strength were determined using the sgRNA Design Tool for Mycobacteria (https://pebble.rockefeller.edu/tools/sgrna-design/). The predicted strength is the prediction of sgRNA effectiveness from 0 (weakest) to 1 (strongest) based on a linear model of sgRNA features. The most important features that determine the predicted iKD strength include the PAM, length of sgRNA targeting sequence, and GC content. One parameter of the sgRNA design for this study was that the predicted iKD strength must be ≥0.90 (Table 1). A second parameter was location of the PAM near the gene start codon, but yet far enough away (at least ≥50 bp distance from the start codon), to avoid a reverse polar effect, a phenomenon in which the repressed gene reduces expression of the upstream genes. The iKD-mmpS4 sgRNA is in the middle of the short gene (417 bp) as there were limited PAM options based on our defined parameters (Table 1). Mabs harboring non-targeting sgRNA (NTsgRNA) and the pLJR962 vector were included as controls.

The sgRNA targeting mmpS4 has the sequence GCCGATCTCAAATTTGACGGACC (SEQ ID NO. 1). The sgRNA targeting mmpL4a has the sequence AGCAAGACCGTGATCGCCAGCCAGC (SEQ ID NO: 2). The sgRNA targeting mmpL4b has the sequence ACCGAGGCCAGCGTCGTATAGAGGA (SEQ ID NO: 3). The sgRNA targeting mps1 has the sequence AGCTTGACGAATGGCCCGCTCAA (SEQ ID NO: 4). The sgRNA targeting mps2 has the sequence GCTCGTCGGGATGAGCCGGGTCGA (SEQ ID NO: 5). The sgRNA targeting gap has the sequence ACATTGACCACCAGCGCACCAAC (SEQ ID NO: 6).

TABLE 1
Mabs^{Sm} ↔ Mabs^{Rg} morphotype switchable strains created with CRISPRi. PAM
sequence, sgRNA targeting sequence, and predicted strength were provided by the sgRNA
design website (https://pebble.rockefeller.edu).

| | | | | | PAM | | | |
|---|---|---|---|---|---|---|---|---|
| | | | | | Location | | | |
| | | | | | from | | | |
| Operon | | sgRNA | | Gene | Gene | sgRNA | sgRNA | |
| and | | Targeting | PAM | Length | Start | Start | End | Predicted |
| Gene | Name | Sequence | Sequence | (bp) | (bp) | (bp) | (bp) | Strength |
| gpl 1 | iKD-mmpS4 | SEQ ID NO: 1 | ACGGAAG | 417 | 244 | 251 | 273 | 0.94 |
| gpl 2 | iKD-mmpL4a | SEQ ID NO: 2 | CGAGAAT | 2880 | 79 | 86 | 110 | 0.99 |
| gpl 3 | iKD-mmpL4b | SEQ ID NO: 3 | TCAGAAC | 2952 | 103 | 110 | 134 | 0.92 |
| gap 1 | iKD-mps1 | SEQ ID NO: 4 | GGAGAAC | 10362 | 136 | 143 | 165 | 0.93 |
| gap 2 | iKD-mps2 | SEQ ID NO: 5 | TCGGAAG | 7743 | 352 | 359 | 382 | 0.90 |
| gap 3 | iKD-gap | SEQ ID NO: 6 | CCAGAAC | 891 | 111 | 118 | 140 | 0.93 |

### Example 2: Mabs^{Sm}↔Mabs^{Rg} Morphotype Switchable Strains Induce the Mabs^{condRg} Morphotype in the Presence of ATc

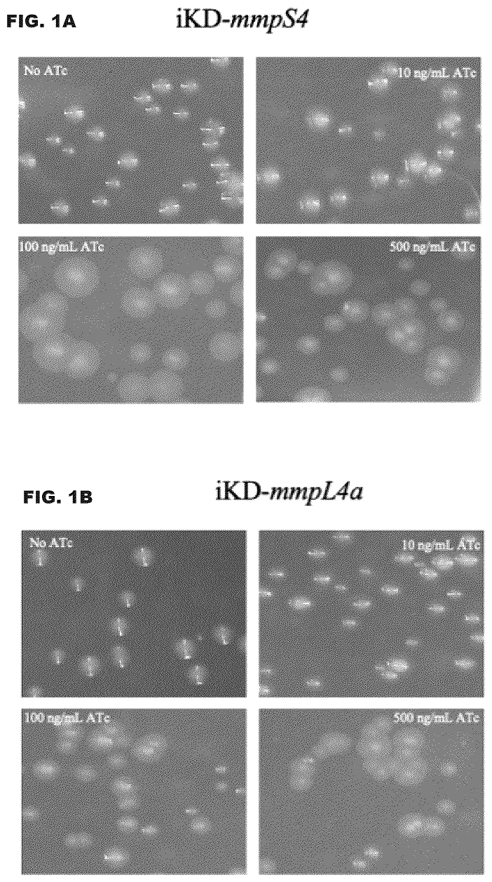

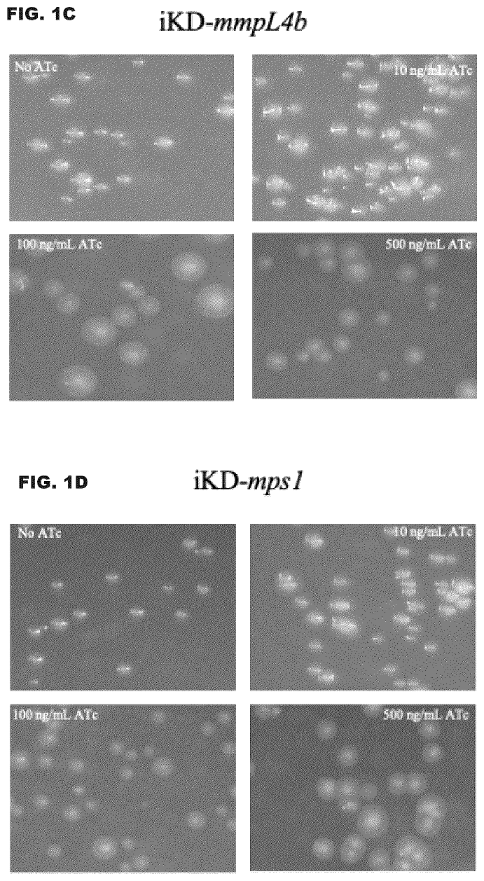

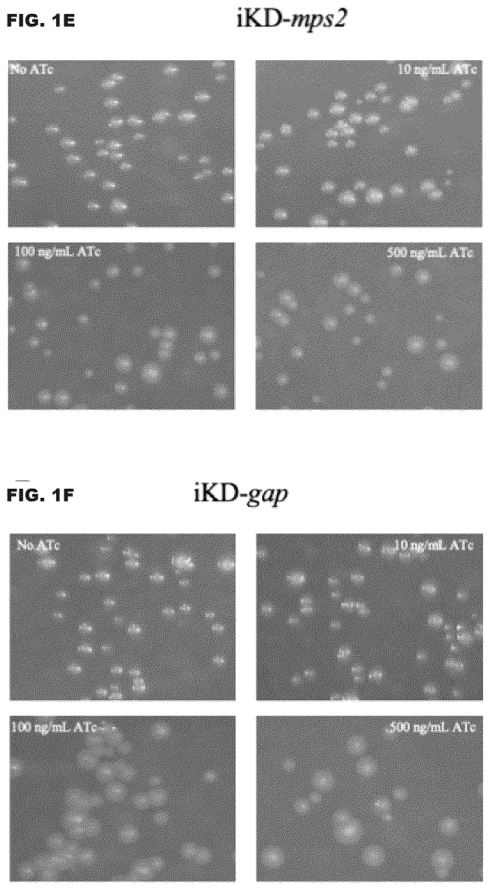

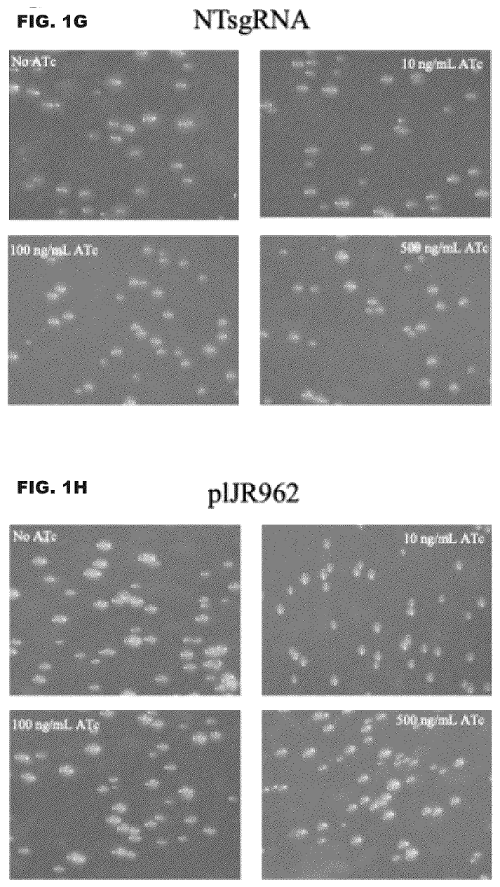

The objective of these experiments was to determine the ATc concentration necessary to induce the Mabs^{Sm}-to-Mabs^{condRg} morphotypic switch within a cell population. The conditional switch for the Mabs^{Sm}↔Mabs^{Rg} morphotype switchable strains was tested in the presence of 10, 100, and 500 ng/mL ATc to induce the CRISPRi-iKD system. The shiny and smooth morphotype characteristics of Mabs^{Sm} colonies and the flat, dried out, rough, and waxy appearance of Mabs^{Rg} colonies were used to differentiate the morphotypes when enumerating CFU (FIGS. 1A-1H). In the presence of 10 ng/mL ATc, the Mabs iKD-mmpL4a, iKD-mmpL4b, and iKD-mps1 strains exhibited 18-39% Mabs conditional rough (Mabs^{condRg}) CFU conversion (FIGS. 2A-2H). The low (10 ng/mL) ATc concentration failed to induce the Mabs^{condRg} morphotype among the CRISPRi-iKD targeted mmpS4, mps2, and gap GPL genes (FIGS. 1A-2H). With 100 ng/mL ATc, Mabs^{condRg} conversion rates were 70-93% among the six Mabs GPL-iKD recombinant strains (FIGS. 2A-2H). The high ATc (500 ng/ml) concentration provided the greatest and most consistent Mabs^{condRg} conversion rate, between 92-96%, for all genes (FIGS. 1A-2H). A non-targeting single guide RNA (NTsgRNA) and Mabs pLJR962 empty vector strain were used as controls and did not convert to the Mabs^{condRg} morphotype (FIGS. 1A-2H). These findings reveal that 500 ng/mL ATc induces the highest percentage Mabs^{condRg} morphotype switching for all morphotype switchable strains.

### Example 3: Mabs^{condRg} Reversion to Mabs^{Sm} in the Absence of ATc

After determining the adequate concentration of ATc necessary for maximal Mabs^{condRg} conversion, Mabs^{condRg} morphotype reversion to Mabs^{Sm} was tested by removing ATc and eliminating GPL CRISPRi-iKD induction. Mabs^{condRg} colonies in the presence of 500 ng/ml ATc were selected and inoculated onto M7H10 agar without ATc. In the absence of ATc, reversion of Mabs^{condRg} to Mabs^{Sm} occurred (FIGS. 3A-3H). These findings demonstrate the reversibility of the morphoswitching strains upon the removal of ATc.

### Example 4: Phenotypic Determination of M. abscessus Morphotype Switching by Aggregation Profiling

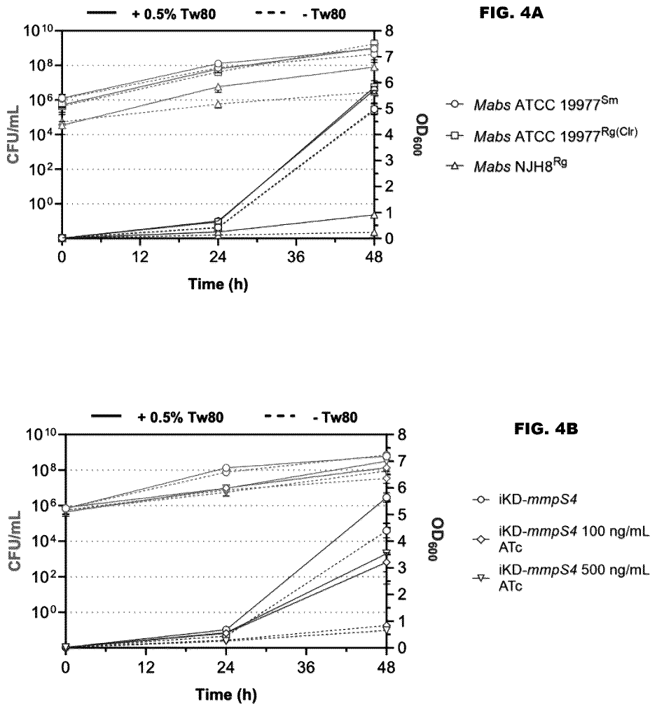

The Mabs^{Sm} and Mabs^{Rg} morphotypes exhibit distinct aggregation patterns in liquid culture, driven by the presence or absence of surface-associated GPLs. Clary et al. established a rapid method for morphotype differentiation based on these aggregation behaviors in the absence of Tween 80 (Tw80), a surfactant commonly used to reduce mycobacterial clumping (Clary et al., “Mycobacterium abscessus Smooth and Rough morphotypes form antimicrobial-tolerant biofilm phenotypes but are killed by acetic acid.” Antimicrob Agents Chemother 2018, 62 (3), e01782-01717). Building on this approach, we used aggregation profiling to assess CRISPRi-mediated morphotype switching from Mabs^{Sm} to Mabs^{condRg}. Viability was not impacted by CRISPRi-mediated repression of target genes (FIGS. 4A and 4B).

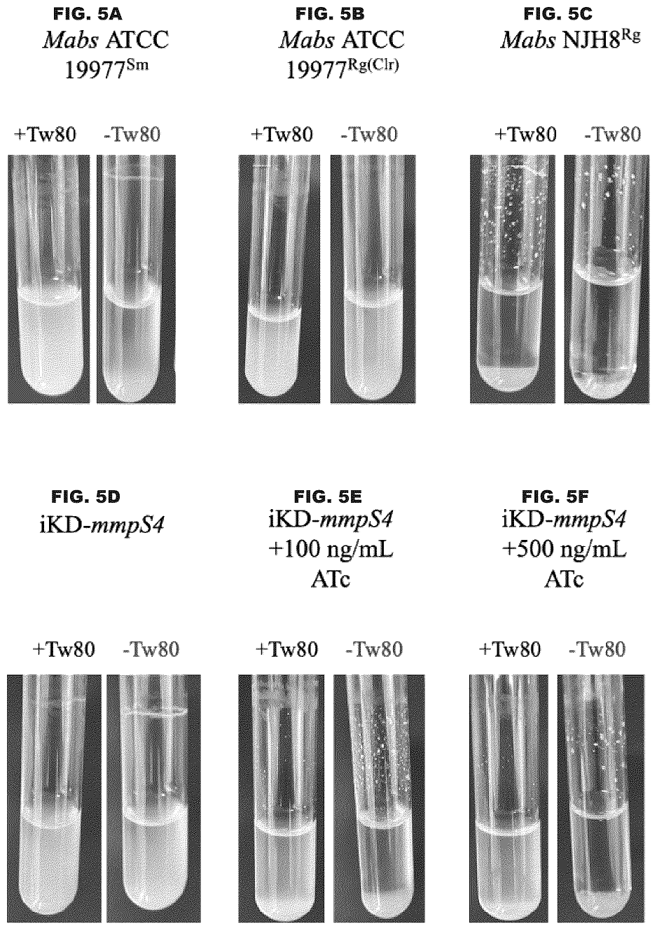

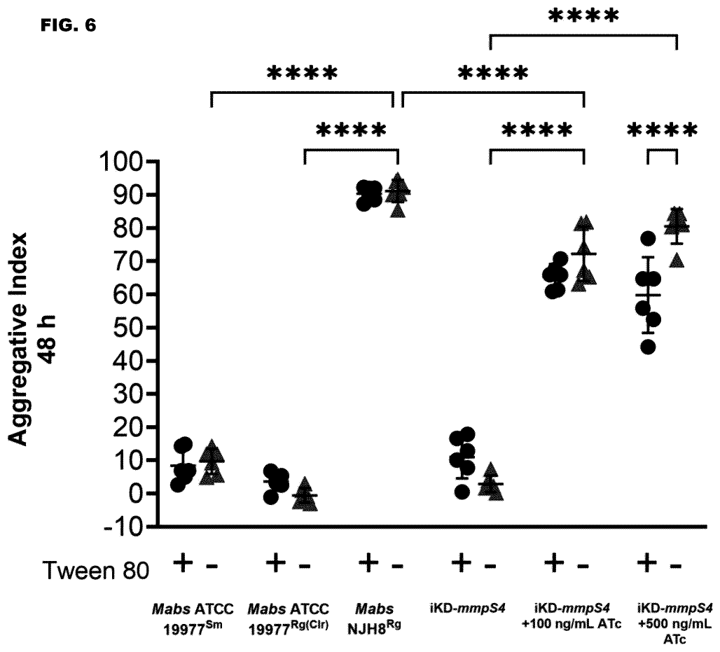

Although aggregation profiling was insufficient for distinguishing Mabs ATCC 19977^{Sm} from its clarithromycin-induced Mabs ATCC 19977^{Rg(Clr)} derivative (FIGS. 5A and 5B), clinical isolate Mabs NJH8^{Rg} exhibited a significantly higher aggregative index (91.14±3.21 vs. ATCC19977S, p<0.001) (FIG. 6). Furthermore, repression of mmpS4 via ATc-inducible CRISPRi led to an ~3-4-fold increase in aggregation relative to uninduced controls (p<0.001), consistent with a switch to the Mabs^{condRg} morphotype (FIG. 6). The presence or absence of Tw80 did not significantly affect aggregative indices in the CRISPRi strain, highlighting the robustness of this phenotypic assay across different culture conditions (FIG. 6).

### Example 5: Mabs^{condRg} Maintenance in the Continued Presence of ATc

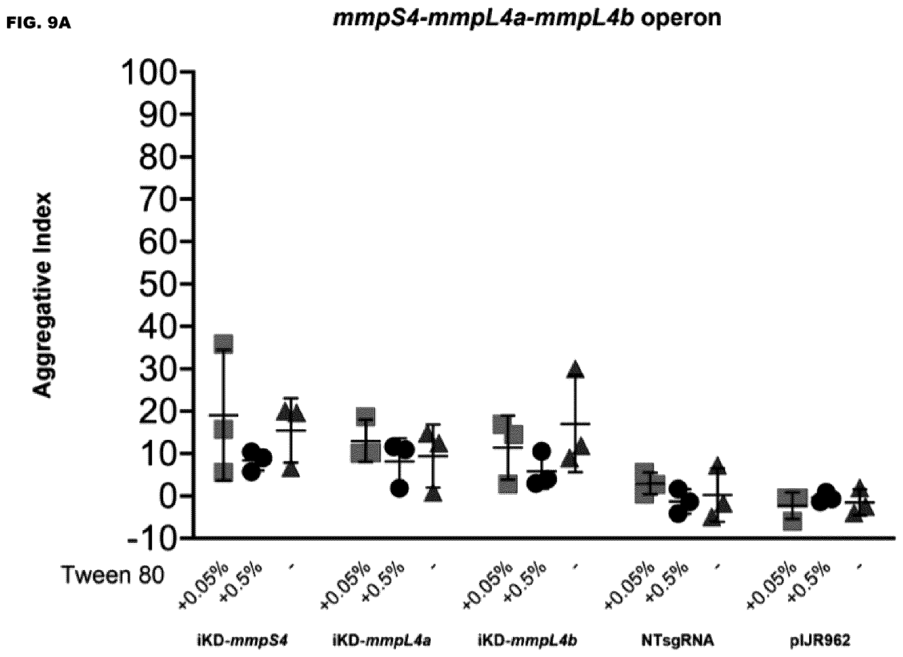

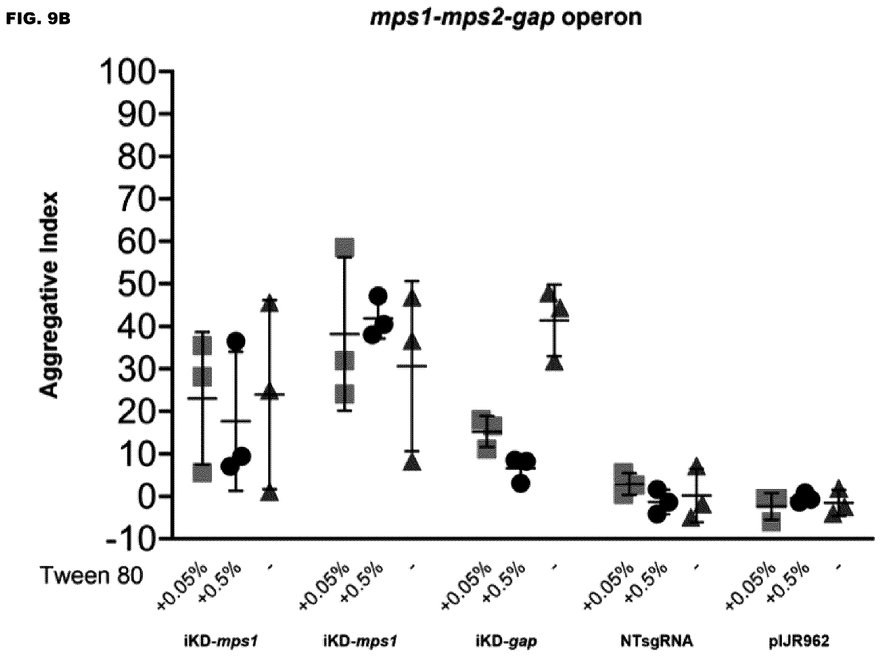

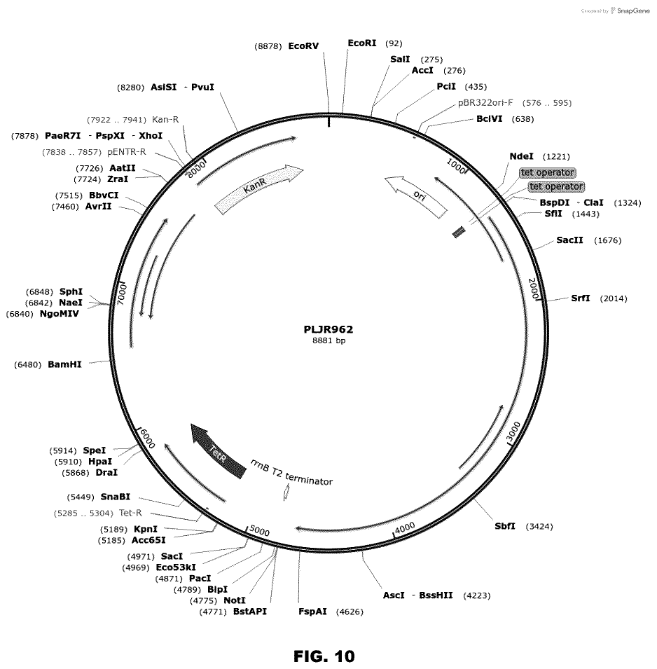

Since the Mabs^{condRg} morphotypic switch reversed to Mabs^{Sm} upon removal of ATc, Mabs^{condRg} maintenance was tested by continuous ATc dosing. Mabs^{condRg} colonies grown on M7H10 agar in the presence of 500 ng/ml ATc were selected and transferred to M7H9 broth and supplemented with ATc every 24 h. After 48 h of growth, aggregation profiling was performed. Mabs^{condRg} aggregation was visibly present immediately after vortexing (FIGS. 7A-7H) with sedimentation occurring after 15 min of static incubation at room temperature (FIGS. 8A-8H). The aggregative index values were inconsistent and generally low (FIGS. 9A and 9B) as the cultures were not grown to the saturation point determined for the previous aggregation profiling (OD₆₀₀ of ~2.5-3.0). Regardless of the low aggregative index values, the visual aggregation of the samples illustrates the retention of the Mabs^{condRg} morphotype in the persistent presence of ATc.

### Example 6: Materials and Methods

Bacterial media and culture conditions. Escherichia coli DH5α was grown in Lysogeny Broth (LB) (10 g tryptone, 5 g yeast extract, 10 g NaCl per L, pH 7.0) and grown on LB agar (pH 7.0). M. abscessus ATCC19977 was cultured in Middlebrook 7H9 medium with 10% ADC (bovine serum albumin, dextrose, catalase), 0.2% glycerol, and 0.05% Tween 80 (Tw80), herein referred to as M7H9 broth. Kanamycin (25 μg/mL) was added to the growth medium when necessary. For growth curves and aggregation assessments, M7H9 broth (10% OADC (oleic acid, bovine serum albumin, dextrose, catalase); 0.2% glycerol, 0.5% Tw80) was utilized. Middlebrook 7H10 (10% ADC; 0.2% glycerol) (M7H10) agar was used for Mabs^{Sm} to Mabs^{condRg} morphotype switch experiments. M7H10 agar with 10% ADS (bovine serum albumin, dextrose, salt) and 0.2% glycerol was used for Mabs^{condRg} to Mabs^{Sm} reversion experiments and growth curves. Liquid cultures were grown at 37 °C on a rotary shaker for 24-48 h. Mabs incubated on M7H10 agar in 12-well plates or 15 mm agar plates were grown for 3 d or approximately 4-5 d, respectively.

Generation of CRISPRi-iKD constructs. Short guide RNAs (sgRNAs) targeted the gpl and gap open reading frame of M. abscessus using the sgRNA Design Tool for Mycobacteria (https://pebble.rockefeller.edu/tools/sgrna-design/). sgRNAs were synthesized by Integrated DNA Technologies™ (Coralville, Iowa, USA), annealed using annealing buffer (50 mM Tris (pH 7.5), 50 mM NaCl, 1 mM EDTA), and cloned into pLJR962 (empty plasmid vector sequence set forth in SEQ ID NO: 7) using BsmBI restriction enzyme cloning. All recombinant pLJR962 plasmids were verified via Sanger sequencing and then electroporated into M. abscessus. All transformants, including the non-targeting sgRNA (NTsgRNA) and pLJR962 vector-only controls were plated on M7H10 agar (10% ADC; 0.2% glycerol; 50 μg/mL kanamycin).

Mabs^{Sm} to Mabs^{condRg} morphotypic switch. Mabs strains containing the CRISPRi guides were grown to mid-logarithmic phase (OD₆₀₀ of 0.6-0.8) in M7H9 broth. Then, cultures were diluted to OD₆₀₀=~0.1, and a 10⁻³ dilution was plated on M7H10 agar containing the following concentrations of anhydrotetracycline (ATc) to induce targeted gene repression: 10, 100, and 500 ng/mL. M7H10 agar was placed in 12-well plates, offering a smaller surface area, Mabs was grown for 3 d, revealing small CFU appropriate for enumeration. Comparably, Mabs grown on M7H10 agar were incubated at 37 °C for approximately 4-5 d prior to CFU quantitation.

Mabs^{condRg} to Mabs^{Sm} reversion. After the Mabs CRISPRi iKD strains were grown on M7H10 agar with ATc, one Mabs^{condRg} colony was selected and added to PBS (1 mL). The sample was vortexed for ~5-10 sec and was plated onto M7H10 agar in 12-well plates without ATc to observe reversion to Mabs^{Sm}. M7H10 agar plates were incubated at 37 °C for 3 d.

Growth curves. Mabs cells from a frozen stock were inoculated into M7H9 broth (3 mL) and grown at 37 °C for 24 h on a rotary shaker. Thereafter, Mabs cultures were diluted to OD₆₀₀ of ~0.01 in M7H9 (3 mL) with and without 0.5% Tw80 and grown at 37 °C in a rotary shaker. The OD₆₀₀ measurements and CFU quantitation were performed at the following times: 0, 24, and 48 h until the desired saturation point was achieved. For Mabs^{Sm}↔Mabs^{Rg} morphotype switchable strains, ATc was supplemented every 24 h. Strains utilized for growth curves include Mabs ATCC 19977^{Sm} and Mabs ATCC 19977^{Rg(Clr)}, which is a Mabs^{Rg} strain that was induced with clarithromycin (Clr). A Mabs^{Rg} clinical isolate, Mabs NJH8^{Rg}, was also utilized due to its Rough morphotype and aggregative capacity.

Aggregation assessment. Mabs aggregation was determined using the aggregation index as described by Clary et al. Mabs cultures were grown at 37 °C for 24 h in a rotary incubator and then diluted to OD₆₀₀ of ~0.01 in M7H9 broth with and without 0.5% Tw80. Mabs cultures were grown at 37 °C on a rotary shaker for 48 h or until cultures reach the desired saturation point. For Mabs^{Sm}↔Mabs^{Rg} morphotype switchable strains, ATc was supplemented every 24 h. Cultures were gently vortexed and the OD₆₀₀ measurement (OD_{T1}) was recorded. Cultures were statically incubated at room temperature for 15 min, followed by a second OD₆₀₀ (OD_{T2}) measurement. The aggregative index was calculated using the equation

$$\frac{{OD}_{T 1} - {OD}_{T 2}}{{OD}_{T 1}} \times 100 .$$

Mabs^{condRg} maintenance. After the Mabs CRISPRi iKD strains were grown on M7H10 agar with 500 ng/ml ATc, five Mabs^{condRg} colonies were selected and added to M7H9 broth (3 mL) with and without 0.05% Tw80 and grown at 37 °C in a rotary shaker for 48 h (with ATc supplementation at 24 h). At 48 h, 0.05% Tw80 cultures were subcultured to OD₆₀₀ of ~0.01 in M7H9 broth with 0.5% Tw80 and grown for 24 h. Cultures were gently vortexed, and the OD 600 measurement (OD_{T1}) was recorded. Cultures were statically incubated at room temperature for 15 min, followed by a second OD₆₀₀ (OD_{T2}) measurement. The aggregative index was calculated using the equation

$$\frac{{OD}_{T 1} - {OD}_{T 2}}{{OD}_{T 1}} \times 100 .$$

Statistical analysis. Statistical analyses were performed using GraphPad Prism 10 (GraphPad Software, San Diego, CA), and p values of <0.05 were considered statistically significant.

### Example 7: Future Experiments

To continue characterization of the morphoswitching strains, qRT-PCR must be completed to quantitatively measure targeted gene knock-down levels of the in the presence of ATc. As polar effects, whereby repression of the first gene in an operon disrupts downstream gene transcription, transcriptional effects on GPL operonic genes must be assessed. PAM location sites were at least ≥50 bp downstream from the gene translational start codon to prevent reverse polar effects. However, in the case of the mbtH-mps1-mps2-gap operon, a reverse polar effect on mbtH expression could occur, in which the repressed gene disrupts expression of the upstream genes due to the targeting sequences being close to the gene start site Therefore, any mbtH transcriptional changes upon mps CRISPRi-iKD will be assessed via qRT-PCR.

Activity of the Mabs^{Sm}↔Mabs^{Rg} morphotype switchable and reversible strains can be further investigated in ex vivo cellular models to assess behavior and responsiveness in host-relevant intracellular environments. Given uncertainty associated with how closely in vitro responses will reflect intracellular dynamics during macrophage infection, parallel testing in infection models will be critical. Additionally, the timing and kinetics of bacterial reversion to Mabs^{Sm} following remain to be characterized. Continuous exposure, such as ATc dosing every ~20 h, will help assess cellular viability and any cytotoxic effects of sustained ATc induction, ensuring compatibility with long-term experiments.

1. A recombinant Mycobacterium abscessus (Mabs) strain expressing a short guide RNA (sgRNA) targeting the gpl open reading frame of Mabs or a sgRNA targeting the gap open reading frame of Mabs.

2. The recombinant Mabs of claim 1, wherein the sgRNA targets a gene selected from the group consisting of: mmpS4, mmpL4a, and mmpL4b.

3. The recombinant Mabs of claim 2, wherein the sgRNA has a sequence set forth in any one of SEQ ID NOs: 1-3.

4. The recombinant Mabs of claim 1, wherein the sgRNA targets a gene selected from the group consisting of: mps1, mps2, and gap.

5. The recombinant Mabs of claim 4, wherein the sgRNA has a sequence set forth in any one of SEQ ID NOs: 4-6.

6. The recombinant Mabs of claim 1, wherein the sgRNA is expressed by a plasmid encoding a CRISPR interference (CRISPRi) inducible knock-down (iKD) system and the CRISPRi-iKD system comprises an inducible TetR-regulated promoter for expressing the sgRNA.

7. A recombinant Mycobacterium abscessus (Mabs) strain comprising a CRISPR interference (CRISPRi) system, wherein the CRISPRi system comprises:
a catalytically inactive Cas9 (dCas9) protein; and
an anhydrotetracycline (ATc)-inducible TetR-regulated promoter operably linked to a sgRNA;
wherein the sgRNA targets a gene selected from the group consisting of mmpS4, mmpL4a, mmpL4b, mps1, mps2, and gap, such that, upon induction with ATc, expression of the sgRNA represses the target gene, inducing a phenotypic morphotype switch from a smooth morphotype (Mabs^{Sm}) to a rough morphotype (Mabs^{condRg}), and in the absence of ATc, the morphotype reverts to Mabs^{Sm}.

8. The recombinant Mabs of claim 7, wherein the sgRNA targets a gene selected from the group consisting of: mmpS4, mmpL4a, and mmpL4b.

9. The recombinant Mabs of claim 7, wherein the sgRNA targets a gene selected from the group consisting of: mps1, mps2, and gap.

10. The recombinant Mabs of claim 7, wherein the sgRNA has a sequence set forth in any one of SEQ ID NOs: 1-6.

11. A plasmid comprising:
a sequence encoding a short guide RNA (sgRNA) configured to target the gpl open reading frame or the gap open reading frame of Mycobacterium abscessus (Mabs); and
a TetR-regulated promoter operably linked to expression of the sgRNA, wherein the plasmid is configured to induce transcriptional repression of the targeted gene upon administration of a tetracycline-class compound.

12. The plasmid of claim 11, wherein the sgRNA targets a gene selected from the group consisting of: mmpS4, mmpL4a, and mmpL4b.

13. The plasmid of claim 12, wherein the sgRNA has a sequence set forth in any one of SEQ ID NOs: 1-3.

14. The plasmid of claim 11, wherein the sgRNA targets a gene selected from the group consisting of: mps1, mps2, and gap.

15. The plasmid of claim 14, wherein the sgRNA has a sequence set forth in any one of SEQ ID NOs: 4-6.

16. A system for producing a reversible morphotype switchable strain of Mycobacterium abscessus (Mabs), comprising:
the plasmid of claim 11; and
a tetracycline-class compound.

17. The system of claim 16, further comprising a Mabs strain.

18. The system of claim 16, wherein the plasmid encoding the CRISPR-iKD system comprises a sequence encoding the sgRNA comprising a sequence set forth in any one of SEQ ID NOs: 1-6.

19. A method of inducing Mycobacterium abscessus (Mabs) morphology change to a rough morphology comprising:
providing a Mabs;
transforming the Mabs with the plasmid of claim 11; and
administering a tetracycline-class compound to the transformed Mabs under conditions sufficient to activate expression of an sgRNA and repress expression of a GPL biosynthesis or transport gene, thereby inducing a rough morphotype in the strain.

20. A method of inducing Mycobacterium abscessus (Mabs) morphology change to a smooth morphology comprising:
providing a transformed Mabs induced to have a rough morphology according to the method of claim 19;
withholding administration and exposure of the transformed Mabs to tetracycline-class compound for at least 18 hours, thereby permitting re-expression of the GPL biosynthesis or transport gene and reversion of the Mabs to a smooth morphology.


SEQUENCE LISTING (7 sequences)

SEQ ID NO: 1 - DNA, 23 bp, Mycobacterium abscessus
  source 1..23: mol_type = other DNA
gccgatctcaaatttgacggacc

SEQ ID NO: 2 - DNA, 25 bp, Mycobacterium abscessus
  source 1..25: mol_type = other DNA
agcaagaccgtgatcgccagccagc

SEQ ID NO: 3 - DNA, 25 bp, Mycobacterium abscessus
  source 1..25: mol_type = other DNA
accgaggccagcgtcgtatagagga

SEQ ID NO: 4 - DNA, 23 bp, Mycobacterium abscessus
  source 1..23: mol_type = other DNA
agcttgacgaatggcccgctcaa

SEQ ID NO: 5 - DNA, 24 bp, Mycobacterium abscessus
  source 1..24: mol_type = other DNA
gctcgtcgggatgagccgggtcga

SEQ ID NO: 6 - DNA, 23 bp, Mycobacterium abscessus
  source 1..23: mol_type = other DNA
acattgaccaccagcgcaccaac

SEQ ID NO: 7 - DNA, 8881 bp, synthetic construct
  source 1..8881: mol_type = other DNA
aataaacgaaaggctcagtcgaaagactgggcctttcgtttatctgttgtttgaaaaaaaaaagcgccgcaactgcggcgcttttttttttgaattctctgaccagggaaaatagccctctgacctggggatttgcgatccctatcagtgatagatataatctgggaggagacgattaatgcgtctcggtttttgtactcgaaagaagctacaaagataaggcttcatgccgaaatcaacaccctgtcattttatggcagggtgttttttttttgtcgacttggggaccctagaggtccccttttttatttttttggatgaagagcattatcaatggctcttcacaggaagcttaaaaaaaaaagccccgcgattgcggggctttttttttttaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccctcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcatggaacaataaacgaaaggctcagtcgaaagactgggcctttcgtttatctgttgtttgaaaaaaaaaagccccgcgattgcggggcttttttttttcatatgtctgaccagggaaaatagccctctgacctggggatttgacttccctatcagtgatagagataatctgggagtccctatcagtgatagagaaggcggtatcgatatgtcggacctggtgctgggcctggccatcggcatcggctcggtgggcgtgggcatcctgaacaaggtgaccggcgagatcatccacaagaactcgcgcatcttcccggccgcccaggccgagaacaacctggtgcgccgcaccaaccgccagggccgccgcctggcccgccgcaagaagcaccgccgcgtgcgcctgaaccgcctgttcgaggagtcgggcctgatcaccgacttcaccaagatctcgatcaacctgaacccgtaccagctgcgcgtgaagggcctgaccgacgagctgtcgaacgaggagctgttcatcgccctgaagaacatggtgaagcaccgcggcatctcgtacctggacgacgcctcggacgacggcaactcgtcggtgggcgactacgcccagatcgtgaaggagaactcgaagcagctggagaccaagaccccgggccagatccagctggagcgctaccagacctacggccagctgcgcggcgacttcaccgtggagaaggacggcaagaagcaccgcctgatcaacgtgttcccgacctcggcctaccgctcggaggccctgcgcatcctgcagacccagcaggagttcaacccgcagatcaccgacgagttcatcaaccgctacctggagatcctgaccggcaagcgcaagtactaccacggcccgggcaacgagaagtcgcgcaccgactacggccgctaccgcacctcgggcgagaccctggacaacatcttcggcatcctgatcggcaagtgcaccttctacccggacgagttccgcgccgccaaggcctcgtacaccgcccaggagttcaacctgctgaacgacctgaacaacctgaccgtgccgaccgagaccaagaagctgtcgaaggagcagaagaaccagatcatcaactacgtgaagaacgagaaggccatgggcccggccaagctgttcaagtacatcgccaagctgctgtcgtgcgacgtggccgacatcaagggctaccgcatcgacaagtcgggcaaggccgagatccacaccttcgaggcctaccgcaagatgaagaccctggagaccctggacatcgagcagatggaccgcgagaccctggacaagctggcctacgtgctgaccctgaacaccgagcgcgagggcatccaggaggccctggagcacgagttcgccgacggctcgttctcgcagaagcaggtggacgagctggtgcagttccgcaaggccaactcgtcgatcttcggcaagggctggcacaacttctcggtgaagctgatgatggagctgatcccggagctgtacgagacctcggaggagcagatgaccatcctgacccgcctgggcaagcagaagaccacctcgtcgtcgaacaagaccaagtacatcgacgagaagctgctgaccgaggagatctacaacccggtggtggccaagtcggtgcgccaggccatcaagatcgtgaacgccgccatcaaggagtacggcgacttcgacaacatcgtgatcgagatggcccgcgagaccaacgaggacgacgagaagaaggccatccagaagatccagaaggccaacaaggacgagaaggacgccgccatgctgaaggccgccaaccagtacaacggcaaggccgagctgccgcactcggtgttccacggccacaagcagctggccaccaagatccgcctgtggcaccagcagggcgagcgctgcctgtacaccggcaagaccatctcgatccacgacctgatcaacaactcgaaccagttcgaggtggacgccatcctgccgctgtcgatcaccttcgacgactcgctggccaacaaggtgctggtgtacgccaccgccaaccaggagaagggccagcgcaccccgtaccaggccctggactcgatggacgacgcctggtcgttccgcgagctgaaggccttcgtgcgcgagtcgaagaccctgtcgaacaagaagaaggagtacctgctgaccgaggaggacatctcgaagttcgacgtgcgcaagaagttcatcgagcgcaacctggtggacacccgctacgcctcgcgcgtggtgctgaacgccctgcaggagcacttccgcgcccacaagatcgacaccaaggtgtcggtggtgcgcggccagttcacctcgcagctgcgccgccactggggcatcgagaagacccgcgacacctaccaccaccacgccgtggacgccctgatcatcgccgcctcgtcgcagctgaacctgtggaagaagcagaagaacaccctggtgtcgtactcggaggaccagctgctggacatcgagaccggcgagctgatctcggacgacgagtacaaggagtcggtgttcaaggccccgtaccagcacttcgtggacaccctgaagtcgaaggagttcgaggactcgatcctgttctcgtaccaggtggactcgaagttcaaccgcaagatctcggacgccaccatctacgccacccgccaggccaaggtgggcaaggacaaggccgacgagacctacgtgctgggcaagatcaaggacatctacacccaggacggctacgacgccttcatgaagatctacaagaaggacaagtcgaagttcctgatgtaccgccacgacccgcagaccttcgagaaggtgatcgagccgatcctggagaactacccgaacaagcagatcaacgacaagggcaaggaggtgccgtgcaacccgttcctgaagtacaaggaggagcacggctacatccgcaagtactcgaagaagggcaacggcccggagatcaagtcgctgaagtactacgactcgaagctgggcaaccacatcgacatcaccccgaaggactcgaacaacaaggtggtgctgcagtcggtgtcgccgtggcgcgccgacgtgtacttcaacaagaccaccggcaagtacgagatcctgggcctgaagtacgccgacctgcagttcgacaagggcaccggcacctacaagatctcgcaggagaagtacaacgacatcaagaagaaggagggcgtggactcggactcggagttcaagttcaccctgtacaagaacgacctgctgctggtgaaggacaccgagaccaaggagcagcagctgttccgcttcctgtcgcgcaccatgccgaagcagaagcactacgtggagctgaagccgtacgacaagcagaagttcgagggcggcgaggccctgatcaaggtgctgggcaacgtggccaactcgggccagtgcaagaagggcctgggcaagtcgaacatctcgatctacaaggtgcgcaccgacgtgctgggcaaccagcacatcatcaagaacgagggcgacaagccgaagctggacttctaaatactctggccatttttctttaccgcctttgagtgagctgataccgctcgccgcagccgaacgaccgagcgcaacgcgtgcggccgctgattagctaagcagaaggccatcctgacggatggcctttttctagaaaaaaaaaaagcgccgcaactgcggcgcttttttttttttaattaaagaaatattggatcgtcggcaccgtcacggccgtgggaggcggcacgatccgcgacgtgatgatcggccgcatccccacggtgctgcgcagtgagctctacgccatcccggcgttgatctgtgcgttcgcacgcacaggcccggtgtgagaagggtctctgacgagcgggagaacccacccggggtgggcgagtttgtcctgcgtgtgctcggtcgagtaggctctgggagtacccgtgtgtacgaccagcacggcatacatcatttcgacgccgagagattcgccgcccgaaatgagcacgatccgcatggtaccatcaggaggaatcacttcgcaatgtctagattagataaaagtaaagtgattaacagcgcattagagctgcttaatgaggtcggaatcgaaggtttaacaacccgtaaactcgcccagaagcttggtgtagagcagcctacattgtattggcatgtaaaaaataagcgggccctgctcgacgccttagccattgagatgttagataggcaccatactcacttttgccctttagaaggggaaagctggcaagattttttacgtaataacgctaaaagttttagatgtgctttactaagtcatcgcgatggagcaaaagtacatttaggtacacggcctacagaaaaacagtatgaaactctcgaaaatcaattagcctttttatgccaacaaggtttttcactagagaacgcgttatatgcactcagcgctgtggggcattttaccttaggttgcgtattggaagatcaagagcatcaagtcgctaaagaagaaagggaaacacctactactgatagtatgccgccattattacgacaagctatcgaattatttgatcaccaaggtgcagagccagccttcttattcggccttgaattgatcatctgcggattagaaaaacaacttaaatgtgaaagtgggtcttaatgaccatgggctagcgctttagctaattaatttaaaataaaaaaggggacctctagggtccccaattaaatagttaacactagtattaaaaaaaaaagccccgcgattgcggggcttttttttttcgacggcgagggttccgaccgctgcaactcccggtgcaaccttgtcccggtctattctcttcactgcaccagctccaatctggtgtgaatgcccctcgtctgttcgcgcaggcggggggctctattcgtttgtcagcatcgaaagtagccagatcagggatgcgttgcaaccgcgtatgcccaggtcagaagagtcgcacaagagttgcagacccctggaaagaaaaatggccagagggcgaaaacaccctctgaccagcggagcgggcgacgggaatcgaacccgcgtagctagtttggaagaatgggtgtctgccgaccacatatatgggccggtcaagataggtttttaccccctctcggctgcatcctctaagtggaaagaaattgcaggtcgtagaagcgcgttgaagcctgagagttgcacaggagttgcaacccggtagccttgttcacgacgagaggagacctagttggcacgtcgcggatggggatcgctgaagactcagcgcagcgggaggatccaagcctcatacgtcaacccgcaggacggtgtgaggtactacgcgctgcagacctacgacaacaagatggacgccgaagcctggctcgcgggcgagaagcggctcatcgagatggagacctggacccctccacaggaccgggcgaagaaggcagccgccagcgccatcacgctggaggagtacacccggaagtggctcgtggagcgcgacctcgcagacggcaccagggatctgtacagcgggcacgcggagcgccgcatctacccggtgctaggtgaagtggcggtcacagagatgacgccagctctggtgcgtgcgtggtgggccgggatgggtaggaagcacccgactgcccgccggcatgcctacaacgtcctccgggcggtgatgaacacagcggtcgaggacaagctgatcgcagagaacccgtgccggatcgagcagaaggcagccgatgagcgcgacgtagaggcgctgacgcctgaggagctggacatcgtcgccgctgagatcttcgtgcactaccggatcgcggcatacatcctggcgtggacgagcctccggttcggagagctgatcgagcttcgccgcaaggacatcgtggacgacggcatgacgatgaagctccgggtgcgccgtggcgcttcccgcgtggggaacaagatcgtcgttggcaacgccaagaccgtccggtcgaagcgtcctgtgacggttccgcctcacgtcgcggagatgatccgagcgcacatgaaggaccgtacgaagatgaacaagggccccgaggcattcctggtgaccacgacgcagggcaaccggctgtcgaagtccgcgttcaccaagtcgctgaagcgtggctacgccaagatcggtcggccggaactccgcatccacgacctccgcgctgtcggcgctacgttcgccgctcaggcaggtgcgacgaccaaggagctgatggcccgcctcggtcacacgactcctaggatggcgatgaagtaccagatggcgtctgaggcccgcgacgaggctatcgctgaggcgatgtccaagctggccaagacctcctgaaacgcaaaaagcccccctcccaaggacactgagtcctaaagaggggggtttcttgtcagtacgcgaagaaccacgcctggccgcgagcgccagcaccgccgctctgtgcggagacctgggcaccagccccgccgccgccaggagcattgccgttcccgccagctagccacctgacgtcgggggggggggaaagccacgttgtgtctcaaaatctctgatgttacattgcacaagataaaaatatatcatcatgaacaataaaactgtctgcttacataaacagtaatacaaggggtgttatgagccatattcaacgggaaacgtcttgctcgaggccgcgattaaattccaacatggatgctgatttatatgggtataaatgggctcgcgataatgtcgggcaatcaggtgcgacaatctatcgcttgtatgggaagccccatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccgggaaaacagcattccaggtattagaagaatatcctgattcaggtgaaaatattgttgatgcgctggcagtgttcctgcgccggttgcattcgattcctgtttgtaattgtccttttaacagcgatcgcgtatttcgcctcgctcaggcgcaatcacgaatgaataacggtttggttgatgcgagtgattttgatgacgagcgtaatggctggcctgttgaacaagtctggaaagaaatgcataatcttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaatcagaattggttaattggttgtaacactggcagagcattacgctgacttgacgggacggcggctttgttgaataaatcgaacttttgctgagttgaaggatcagatcacgcatcttcccgacaacgcagaccgttccgtggcaaagcaaaagttcaaaatcaccaactggtccacctacaacaaagctctcaccaaccgtggctccctcacgatatc